(19) United States
(12) Patent Application Publication
(10) Pub. No.: US 2024/0000726 A1
(43) Pub. Date: Jan. 4, 2024

(54) COMPOUNDS AND METHODS FOR SCAVENGING DICARBONYL ELECTROPHILES

(71) Applicants: VANDERBILT UNIVERSITY, Nashville, TN (US); The United States as Represented by The Department of Veterans Affairs, Washington, DC (US)

(72) Inventors: Keith T. Wilson, Nashville, TN (US); John A. Oates, Nashville, TN (US); Olivier Boutaud, Nashville, TN (US); Venkataraman Amarnath, Nashville, TN (US); Erica J. Carrier, Nashville, TN (US); Alain P. Gobert, Nashville, TN (US)

(21) Appl. No.: 18/220,137

(22) Filed: Jul. 10, 2023

Related U.S. Application Data
(63) Continuation-in-part of application No. 16/893,425, filed on Jun. 4, 2020, now Pat. No. 11,696,903.
(60) Provisional application No. 62/857,165, filed on Jun. 4, 2019.
(60) Provisional application No. 62/978,183, filed on Feb. 18, 2020.

(51) Int. Cl. A61K 31/135; A61P 35/00

(52) U.S. Cl. CPC A61K 31/135; A61P 35/00

(57) ABSTRACT

Compounds and methods of scavenging bifunctional electrophiles and reducing the occurrence of lysyl-levuglandin adducts in a subject in need thereof by administering a levuglandin adduct formation inhibiting amount of a compound of the following formula:

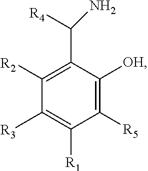

wherein the variables are defined herein.

### PRIOR APPLICATIONS

This application is a continuation-in-part of U.S. patent application Ser. No. 16/893,425, filed Jun. 4, 2020, now allowed, which claims benefit to U.S. Patent Application No. 62/857,165, filed Jun. 4, 2019; and U.S. Patent Application No. 62/978, 183, filed Feb. 18, 2020. The contents of these applications are incorporated herein by reference.

### GOVERNMENT SUPPORT

This invention was made with government support under P30ES000267, P50CA090949, R₂₁CA201856, PO1CA028842, PO1CA116087, R₀₁CA190612, R₀₁DK053620, P30DK058404, P50GM015431 and 6R₂₁CA187495 awarded by the National Institutes of Health and grant number W81XWH-18-1-0301 awarded by the Department of Defense. The government has certain rights in the invention.

### FIELD OF THE INVENTION

Embodiments of this invention relate to methods of inhibiting the modification of histones and DNA by levuglandins in a subject in need thereof by administering a compound of the present invention or a pharmaceutically acceptable salt thereof.

Embodiments of this invention also are scavengers of bifunctional electrophiles, including bifunctional electrophiles that are generated in vivo during carcinogenesis.

Embodiments of this invention also relate to inhibiting the formation of levuglandin adducts.

Embodiments of this invention also relate to inhibiting the development of intramucosal carcinomas, as well as reducing the dysplasia that is a carcinoma precursor.

Embodiments of this invention also relate to methods of treating gastrointestinal cancer.

### SUMMARY OF THE INVENTION

The burden of colorectal cancer (CRC) in the United States and worldwide is massive, representing the 3rd most common cancer and 2nd most cause of cancer deaths. The etiology of CRC is multifactorial and encompasses genetic factors, environmental exposures, and/or inflammation. Despite many decades of investigation, the molecular process by which healthy colonic epithelial cells transform remains largely undetermined. In patients with inflammatory bowel disease the risk for cancer is especially high, leading to colitis-associated carcinoma (CAC). The strong mucosal immune response that occurs during colitis leads to the generation of effector molecules, including reactive oxygen species and prostaglandins, which are involved in the formation of dicarbonyl electrophiles, such as levuglandins or malondialdehyde. These electrophiles are highly reactive with DNA and lysine residues, notably in histones, thus favoring mutagenesis and somatic genomic abnormalities. However, the role of endogenous electrophiles in colorectal carcinogenesis remains unknown despite the fact that they can be mutagenic. The present inventors have discovered that electrophiles are involved in colon carcinogenesis: 1) The electrophile protein adducts on lysines are elevated in: i) human tissues from ulcerative colitis (UC), UC dypsplasia, and CAC with a progressive increase; ii) colonic tumors of CRC patients; iii) dyplastic tumors of C57BL/6 mice treated with azoxymethane-dextran sulfate sodium (AOM-DSS), a model of CAC, compared to non-tumor areas; iv) dysplastic tumors of mice with colon-specific homozygous deletion of Apc; 2). The present inventors show that compounds of the present invention are potent scavengers that react with dicarbonyl electrophiles, and inhibit adduct formation. One compound of the present invention, 2-hydroxybenzylamine (2-HOBA) is a natural product that has successfully completed Phase I human testing at Vanderbilt University and has been shown to protect mice from oxidative damage in models of hypertension and Alzheimer's disease. Another compound of the present invention, 5-ethyl-2-hydroxybenzylamine (EtHOBA) is an analog of HOBA, which can penetrate the nucleus and protect nuclear proteins from electrophilic oxidation. 3) In the AOM-DSS model, treatment of mice with EtHOBA significantly reduces adduct formation, tumor development and dysplasia, while enhancing antitumoral immune response. The present inventors discovered that electrophiles have a key role in colon carcinogenesis via genomic instability, epigenetic dysregulation, and/or suppression of antitumoral immunity, and are key targets for cancer prevention.

Inflammation and subsequent cyclooxygenase-2 (COX-2) activity has long been linked with the development of cancer, although little is known about any epigenetic effects of COX-2. A product of COX-2 activity, levuglandin (LG) quickly forms covalent bonds with nearby primary amines, such as those in lysine, which leads to LG-protein adducts. Here, the present inventors demonstrate that COX-2 activity causes LG-histone adducts in cultured cells and liver tissue, detectable through LC/MS, with the highest incidence in histone H4. Adduction is blocked by a reactive dicarbonyl scavenger, which has no effect on COX-2 activity as measured by PGE₂ production. Formation of LG-histone adduct is associated with an increased histone solubility in NaCl, indicating destabilization of the nucleosome structure; this is also reversed with scavenger treatment. These data demonstrate that COX-2 activity can cause histone adduction and loosening of the nucleosome complex, which could lead to altered transcription and contribute to carcinogenesis.

Additionally, Helicobacter pylori (Hp) (H. pylori) induces an innate immune response in epithelial and myeloid cells that leads to inflammation-associated cancer. The inflammatory effector molecules, such as prostaglandins and reactive oxygen species, can generate the bifunctional electrophiles: levuglandins (LG), malondialdehyde, 4-oxo-nonenal, and acrolein. These molecules form covalent adducts on DNA bases and on lysines (Lys-LG) in histones, thus disrupting DNA/histone interactions and increasing risk for mutations. The present inventors have discovered that compounds of the present invention scavenge said electrophiles, which prevents adduct formation.

Accordingly, embodiments of the present invention include compounds and methods for scavenging bifunctional electrophiles and/or LG-lysine adducts and/or lysyl-LG adducts in a patient in need thereof.

Gastric cancer (GC) is the fourth leading cause of cancer death worldwide with over one million new cases per year. The intestinal type of GC has an inflammatory etiology, which is initiated by the pathogen Helicobacter pylori. This bacterium infects half of the world's population and causes universal non-atrophic gastritis that can progress to the precancerous lesions of multifocal atrophic gastritis, intestinal metaplasia, and dysplasia, and then on to gastric adenocarcinoma. Although eradication of H. pylori results in attenuated progression to GC, it does not necessarily reduce cancer risk once precancerous lesions are present. In addition, antibiotic resistance and reinfection rates affect the efficacy of antibiotic therapies. Screening upper endoscopy is a frequent strategy in some high risk regions to prevent GC, but this is far from universally applied. In this context, alternative strategies to prevent carcinogenesis may positively impact H. pylori-infected patients, especially those with precancerous lesions.

The mucosal innate immune response of the infected stomach leads to the formation of prostanoids, reactive oxygen species, and nitrogen compounds derived from the L-arginine-nitric oxide metabolic pathway. These primary molecules are highly reactive and undergo further reactions to form products of lipid peroxidation, termed dicarbonyl electrophiles. These include isolevuglandins, malondialdehyde, 4-hydroxy-nonenal, 4-oxo-nonenal, methylglyoxal, and acrolein, which is also generated by β-elimination from 3-aminopropanal. These strong oxidants react principally with hard nucleophiles, such as amines present in nucleic acid bases and lysine residues, and form irreversible covalent adducts, which may lead to changes in cell signaling, somatic genomic abnormalities and epigenetic alterations. Of importance, we have reported that gastric epithelial cells of patients with precancerous lesions exhibit high levels of nuclear adducts of isolevuglandins to lysine. Moreover, we have shown that the treatment of transgenic insulin-gastrin (INS-GAS) mice with an experimental scavenger of electrophiles prevented H. pylori-induced DNA damage, somatic mutations, and development of gastric carcinoma.

The present inventors have discovered that compounds of the present invention react with all electrophiles at a rate 3 orders of magnitude faster than with lysine, thus preventing adduct formation with macromolecules. Therefore, compounds of the present invention are well positioned as a clinically available chemopreventive agent for the development of gastric cancerous lesions.

Thus, one embodiment of the present invention is a method of preventing, treating, or ameliorating gastric cancer.

Another embodiment is a method of inhibiting formation of levuglandin adducts of histone and DNA in a subject in need thereof by administering a compound of the present invention or a pharmaceutically acceptable salt thereof.

Another embodiment of the present invention is a method of treating pre-malignant lesions by administering a compound of the present invention or a pharmaceutically acceptable salt thereof.

Another embodiment of the present invention is a method of scavenging levuglandins in nucleus of cells by administering a compound of the present invention or a pharmaceutically acceptable salt thereof.

Another embodiment of the present invention is preventing malignant mutations of pre-cancerous conditions by administering a compound of the present invention or a pharmaceutically acceptable salt thereof.

Another embodiment of the present invention is decreasing a subject's risk of developing cancer by administering a compound of the present invention or a pharmaceutically acceptable salt thereof.

Another embodiment of the present invention is the prevention of cellular transformation to malignancy by administering a compound of the present invention or a pharmaceutically acceptable salt thereof.

Another embodiment of the present invention is a method preventing further mutations in colon, esophagus, breast, lung, pancreas, gastrointestinal cancer, and/or prostate cancers in a subject in need thereof by administering a compound of the present invention or a pharmaceutically acceptable salt thereof.

Another embodiment of the present invention is a method treating and/or preventing a disorder resulting from elevated levels of LG-histone adduct formation and adducts on DNA bases and on lysines (Lys-LG) in histones.

Another embodiment of the present invention is when the disorder resulting from elevated levels of LG-histone adduct formation is neoplasia. In other embodiments, the neoplasia is a brain cancer, a bone cancer, an epithelial cell-derived neoplasia (epithelial carcinoma), a basal cell carcinoma, an adenocarcinoma, a gastrointestinal cancer, a lip cancer, a mouth cancer, an esophageal cancer, a small bowel cancer, a stomach cancer, a colon cancer, a liver cancer, a bladder cancer, a pancreas cancer, an ovary cancer, a cervical cancer, a lung cancer, a breast cancer, a skin cancer, a squamus cell cancer, a basal cell cancer, a prostate cancer, a renal cell carcinoma, a cancerous tumor, a growth, a polyp, an adenomatous polyp, a familial adenomatous polyposis or a fibrosis resulting from radiation therapy.

Another embodiment of the present invention relates to treatment of a disease such as especially pre-malignant lesions of the gastrointestinal tract, colon or esophagus (Barrett's esophagus) or a colon cancer or other malignancies, preferably pre-malignant colon lesions or a colon cancer, in a subject in need thereof. The other malignancies to be treated according to the present invention are preferably selected from the group consisting of breast cancer, lung cancer, ovarian cancer, lymphoma, head and neck cancer and cancer of the esophagus, stomach, bladder, prostrate, uterus and cervix.

Another embodiment of the present invention is a method of inhibiting the progression of a gastrointestinal cancer in a subject, comprising administering to the subject a levuglandin adduct formation inhibiting amount of a compound of the following formula:

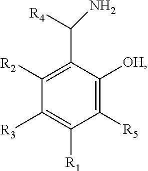

wherein: R₁ is H, D, D₂, substituted or unsubstituted alkyl, substituted or unsubstituted alkoxy, carboxyl, substituted or unsubstituted aryl, or substituted or unsubstituted cycloalkyl; R₂ is H, substituted or unsubstituted alkyl, substituted or unsubstituted alkoxy, alkyl-alkoxy-R₄; R₃ is H, halogen, substituted or unsubstituted alkyl, substituted or unsubstituted alkoxy, hydroxyl, nitro; R₄, H, D, D₂, substituted or unsubstituted alkyl, substituted or unsubstituted alkoxy, carboxyl, substituted or unsubstituted aryl, or substituted or unsubstituted cycloalkyl; R₅ is H, substituted or unsubstituted alkyl, substituted or unsubstituted alkoxy; and pharmaceutical salts thereof.

In one aspect of this embodiment, the cancer is colorectal cancer.

In another aspect of this embodiment, the compound is 2-hydroxybenzylamine, methyl-2-hydroxybenzylamine, or ethyl-2-hydroxybenzylamine.

Another embodiment of the present invention is a method of mitigating the progression of pre-malignant lesions in a subject in need thereof by administering an effective amount of a compound of the following formula:

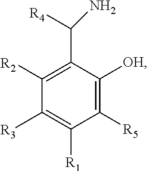

wherein: R₁ is H, D, D₂, substituted or unsubstituted alkyl, substituted or unsubstituted alkoxy, carboxyl, substituted or unsubstituted aryl, or substituted or unsubstituted cycloalkyl; R₂ is H, substituted or unsubstituted alkyl, substituted or unsubstituted alkoxy, alkyl-alkoxy-R₄; R₃ is H, halogen, substituted or unsubstituted alkyl, substituted or unsubstituted alkoxy, hydroxyl, nitro; R₄ H, D, D₂, substituted or unsubstituted alkyl, substituted or unsubstituted alkoxy, carboxyl, substituted or unsubstituted aryl, or substituted or unsubstituted cycloalkyl; R₅ is H, substituted or unsubstituted alkyl, substituted or unsubstituted alkoxy; and pharmaceutical salts thereof.

In another aspect of this embodiment, the lesions are in a gastrointestinal tract.

Another embodiment of the present invention is a method of treating gastrointestinal carcinoma in a subject in need thereof, comprising administering an effective amount of a compound of the following formula:

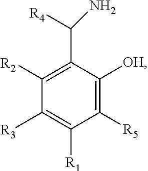

wherein: R₁ is H, D, D₂, substituted or unsubstituted alkyl, substituted or unsubstituted alkoxy, carboxyl, substituted or unsubstituted aryl, or substituted or unsubstituted cycloalkyl; R₂ is H, substituted or unsubstituted alkyl, substituted or unsubstituted alkoxy, alkyl-alkoxy-R₄; R₃ is H, halogen, substituted or unsubstituted alkyl, substituted or unsubstituted alkoxy, hydroxyl, nitro; R₄ H, D, D₂, substituted or unsubstituted alkyl, substituted or unsubstituted alkoxy, carboxyl, substituted or unsubstituted aryl, or substituted or unsubstituted cycloalkyl; R₅ is H, substituted or unsubstituted alkyl, substituted or unsubstituted alkoxy; and pharmaceutical salts thereof.

In aspects of this embodiment, the lesion exists in the colon, esophagus, breast, lung, pancreas, gastrointestinal tract, and/or prostate.

In other aspects, the inhibition step lowers levels of LG-histone adduct formation in said subject.

In other aspects, the subject was first diagnosed with an H. pylori infection.

Another embodiment of the present invention is a commercial package or product comprising an adduction inhibitor, in particular those mentioned herein, or a pharmaceutically acceptable salt thereof, together with instructions for the treatment of a disease such as especially gastrointestinal cancer, pre-malignant gastrointestinal lesions, colon lesions or a colon cancer or other malignancies, preferably pre-malignant colon lesions or a colon cancer, in subject in need thereof.

According to the present invention, a patient is treated with therapeutically effective amounts of an adduction inhibitor of the present invention, each according to a dosage regimen that is appropriate for the individual agent. For example, the adduction inhibitor may be administered once or more daily, on alternate days or on some other schedule—as is appropriate. One of skill in the art has the ability to determine appropriate pharmaceutically effective amounts of the combination components.

In the context of the present invention the terms “treatment” or “treat” refer to both prophylactic or preventative treatment as well as curative or disease modifying treatment, including treatment of patients at risk of contracting the disease or suspected to have contracted the disease as well as patients who are ill or have been diagnosed as suffering from a disease or medical condition.

### BRIEF DESCRIPTION OF THE DRAWINGS

FIG. 1 shows a scheme and structure of the LG-lysyl adduct and the fragment ions monitored in positive ion mode (+H).

FIG. 2 shows immunohistochemistry for LG-protein adducts, human colon TMA. Scale bar, 50 μm.

FIGS. 3A and 3B show data from C57BL/6 mice were treated or not with various concentrations of EtHOBA. (FIG. 3A) Body weight was assessed every day. (FIG. 3B) After 6 days, mice were euthanized and EtHOBA was measured in the colon. n=5 mice/group.

FIG. 4 shows immunohistochemistry for LG-protein adducts. Scale bar, 50 μm.

FIGS. 5A, 5B, 5C, 5D, 5E, 5F, and 5G show mice (n=10-12/group)±AOM-DSS±1.5 mg/ml EtHOBA. (FIG. 5A) Body weight; ″P<0.05 compared to AOM-DSS-treated mice. (FIG. 5B) Tumor number. (FIG. 5C) Average tumor size per mouse. (FIG. 5D) Tumor burden. (FIG. 5E) Number of adenomas. (FIG. 5F) Frequency of diagnoses; ND, no dysplasia. (FIG. 5G) H&E staining; each arrow depicts a tumor. Scale bar, 50 μm.

FIG. 6 shows representative data of levels of lysyl-L G adducts in the colon of CRC patients. Adducts isolated from tumor (T) or non-tumor (NT) biopsies were analyzed by LC/ESI/MS/MS.

FIGS. 7A, 7B, and 7C show data related to CDX2P-CreER^{T2}; Apc^{fl/fl} mice (n=5) treated with 25 mg/kg tamoxifen. At day 33, animals were euthanized. (FIG. 7A) Colon, from proximal (left) to rectum (right), showing tumors (arrows). (FIG. 7B) H&E staining; each arrow depicts a tumor. Lower panel, high power view showing high grade dysplasia. (FIG. 7C) IHC for L G-protein adducts. Scale bar, 50 μm.

FIG. 8 shows immune cell infiltration score in AOM-DSS-treated mice±EtHOBA.

FIG. 9 shows gene expression in the colon tissues of mice treated or not with AOM-DSS±EtHOBA.

FIG. 10 shows a representative IF for CD₆₈₊NOS2+ cells in tumors of AOM-DSS-treated mice±EtHOBA (n=3); NOS2; CD_{68;} DAPI. Scale bar, 50 μm.

FIGS. 11A, 11B, 11C, 11D, 11E, and 11F are a series of graphs showing that LG-lysine adducts of histones are found in cells and tissue, dependent on COX-2 activity. RAW264.7 mouse macrophage (FIG. 11A) and A549 human lung carcinoma (FIG. 11C) cells were stimulated to express COX-2, then given 20 μM arachidonic acid (AA) or vehicle. A subgroup of cells was preincubated 45 min with 50 μM indomethacin. As a measure of COX activity, PGE₂ was determined by GC/MS from cell media prior to lysis (FIG. 11B and FIG. 11D). Nuclei were isolated, and histones were extracted and digested to individual amino acids prior to LC/ESI/MS/MS analysis. *, p<0.05; ***, p<0.001 by ANOVA followed by Tukey's post-test (n≥5). (FIG. 11E). Histones were extracted from nuclei of rat liver, and analyzed as above for LG-lactam adduct. COX-2 protein was analyzed by Western blotting and plotted against lactam adduct levels. Each point corresponds to 1 liver, and shown is the line of regression (r²=0.7237). Pearson r=0.8507; two-tailed p=0.0152. (FIG. 11F) LC-MS chromatograph of histones isolated from a rat liver with relatively high COX-2 expression (COX-2 band intensity of 117 arbitrary units).

FIGS. 12A, 12B, 12C, 12D show LG-lysyl adducts are predominantly detected on histone H4. (FIG. 12A). A Ponceau stain of a sample A549 histone extraction is shown, along with band identities. Histones were extracted from nuclei in 0.4N H₂SO₄, resolved on 4-12% SDS-PAGE gradient gel and transferred to nitrocellulose. H3 and H2B tend to run together as one band. (FIG. 12B). RAW264.7 or A549 cells were stimulated to express COX-2 and given 20 μM ¹⁴C-AA for 1 h. Cells were lysed, nuclei were isolated, and histones extracted, concentrated, and resolved on SDS-PAGE prior to transferring to nitrocellulose and exposing to film. Shown is the Coomassie stain of the SDS-PAGE gel (left) and the result of autoradiography (right). The present inventors have observed that Ponceau, Coomassie, and silver stains each preferentially detect different histone or acid-soluble proteins. (FIG. 12C-FIG. 12D). RAW264.7 (FIG. 12C) or A549 (FIG. 12D) cells were stimulated to express COX-2, and treated with 20 μM AA for 1 h prior to histone extraction. 350-400 μg of total histone was loaded onto 4-12% SDS-PAGE gel and transferred to nitrocellulose. Individual bands were excised horizontally and proteins digested directly off the nitrocellulose by serial incubations with Pronase and aminopeptidase. The results were analyzed by LC/ESI/MS/MS, and the chromatographs of the H3/H2B and H4 bands shown against the LG-lysyl internal standard. The H2A chromatograph is shown as a representative negative result; no co-migrating peaks were seen in any other bands.

FIGS. 13A, 13B, and 13C show that the scavenger EtSA blocks LG-lysyl adduct formation in RAW264.7 and A549 histones, without affecting COX-2 activity. (FIG. 13A). Scavengers were screened in RAW264.7 cells for the ability to decrease LG adduct formation on histones. Scavengers used were glucosamine (GA), 3-methoxysalicylamine (3-MoSA), pentylpyridoxamine (PPM), and 5-ethylsalicylamine (EtSA). Cells were stimulated to express COX-2, pretreated 45 min. with 500 μM scavenger or vehicle (H2O), and given 20 μM AA for 1 h before lysing and extracting histones. Histone proteins were analyzed by LC/ESI/MS/MS for LG-lysyl lactam adduct, n=2. (FIG. 13B) Stimulated A549 cells were pretreated with 30, 300, or 1000 μM EtSA prior to 1 h with 20 μM AA, and histones analyzed for LG-lysyl adduct. *, p<0.05 by one-way ANOVA followed by Dunnett's multiple comparisons post-test (n=3-5). (FIG. 13C). A549 cells were stimulated, pretreated 45 min. with 1000 μM EtSA or H₂O vehicle, and given 20 μM AA for 1 h. Media was analyzed by GC/MS for PGE₂ (n=3). There was no effect on PGE₂ production at lower doses of EtSA (data not shown).

FIGS. 14A and 14B. LG-lysyl adduct formation on histone H4 decreases DNA-histone interaction. A549 cells were stimulated and given DMSO vehicle (C lanes) or 20 μM AA for 1 h (A lanes). A subgroup of cells was treated with 500 μM EtSA 45 min prior to adding AA (E lanes). Nuclei were extracted with 0.6, 0.9, or 1.2 M NaCl buffer, and the supernatant evaluated by Western blotting for histone H4. Shown is a representative Western blot (FIG. 14A) as well as the pooled results of 4 experiments (FIG. 14B). Different exposure times may have been used for the 0.9 M and 1.2 M bands. ***, p<0.001 by one-way ANOVA followed by Tukey's multiple comparisons post-test. NS, not significant.

FIGS. 15A, 15B, and 15C are graphs showing C57BL/6 (FIG. 15A) or INS-GAS mice (FIG. 15B) were infected for 8 weeks with H. pylori PMSS1. Lysyl-LG adducts were measured in the gastric tissues by LC/ESI/MS/MS. *P<0.05. (FIG. 15C) Lysyl-LG adducts concentration was determined in AGS cells infected (plain bars) or not (open bars) with H. pylori PMSS1 for 24 h, in the presence or absence of EtSA. *P<0.05 vs. uninfected cells. § P<0.05 vs. infected cells without EtSA.

FIGS. 16A, 16B, 16C, 16D, 16E, and 16F show INS-GAS mice (8 per group) were infected or not with H. pylori strain PMSSI for 8 weeks and then treated with EtSA (7.5 mg/ml) one week after infection. (FIG. 16A) EtSA concentration in gastric tissues; **P<0.01, ****P<0.0001 vs. mice without EtSA. (FIG. 16B) Frequency of diagnoses. IMC, intramucosal carcinoma; LGD, low grade dysplasia; ND, no dysplasia. (FIG. 16C) Quantification of extent of dysplasia and cancer as a percentage of tissue sections. (FIG. 16D) H&E staining of INS-GAS mouse stomach tissues, showing carcinoma in an infected mouse; scale bar, 50 μm. In B and C, *P<0.05, **P<0.01 vs. infected mice.

FIG. 17 is a graph showing reduction in the concentration of lysyl-levuglandin adducts in the stomachs of H. pylori infected mice treated with 2-hydroxybenzylamine for 8 weeks.

FIG. 18A, 18B is a graph showing measurement of 2-HOBA in the gastric tissues. (FIG. 18A) FVB/N INS-GAS mice were infected two times, or not, with H. pylori PMSS1. Seven days after the first infection, 2-HOBA (1 mg/ml or 3 mg/ml) was given continuously in the drinking water. After 56 days, animals were euthanized, and the stomach was removed. (FIG. 18B) The gastric concentration of 2-HOBA was measured by LC/ESI/MS/MS. P was determined by one-way ANOVA and Tukey test. Control, n=5; 2-HOBA (3 mg/ml), n=5; H. pylori, n=5; H. pylori+2-HOBA (1 mg/ml), n=5; H. pylori+2-HOBA (3 mg/ml), n=13.

FIG. 19A, 19B, 19C, 19D, 19E shows the Effect of 2-HOBA on H. pylori-mediated diseases. (FIG. 19A) Mice were infected and treated as depicted in FIG. 1A. After 56 days, colonization of the stomach of INS-GAS by H. pylori was assessed by serial dilution and culture. (FIG. 19B) Longitudinal sections were stained by H&E. The black and yellow arrows show areas of stromal reaction and epithelial neoplastic cells infiltrating the lamina propria in the stromal reaction, respectively; the scale bars represent 100 μm. (FIG. 19C-FIG. 19E) Histologic gastritis (FIG. 19C), the frequency of LGD and IMC in infected mice (FIG. 19D) and the extent of dysplasia and cancer (FIG. 19E) were determined by scoring H&E staining. ND, no dysplasia. P was calculated by one-way ANOVA using Tukey test (FIG. 19C) or Dunnett's multiple comparisons test (FIG. 19E) and by Chi-square test (FIG. 19D). Control, n=8; 2-HOBA (3 mg/ml), n=8; H. pylori, n=20; H. pylori+2-HOBA (1 mg/ml), n=20; H. pylori+2-HOBA (3 mg/ml), n=19.

FIG. 20 is a graph showing correlation plots comparing extent of dysplasia and cancer to 2-HOBA concentration in the gastric tissues. Statistical analysis was performed using the one-tailed Pearson correlation test. The dotted lines denote the 90% confidence interval of the best-fit line. Each dot represents a mouse.

FIG. 21 is a set of graphs showing the effect of 2-HOBA on H. pylori-induced gastric immune response. The expression of the genes Il1b, Tnf, Nos2, Cxcl1, Il17, and Ifng was analyzed by RT-real time PCR using RNA isolated from the gastric tissues of INS-GAS mice infected or not with H. pylori±2-HOBA. P was determined by ANOVA and Dunnett's test. The number of mice per group that were analyzed were: Control, n=5; 2-HOBA (3 mg/ml), n=5; H. pylori, n=11; H. pylori+2-HOBA (1 mg/ml), n=11; H. pylori+2-HOBA (3 mg/ml), n=13.

FIG. 22A, 22B, 22C, 22D shows DNA damage in INS-GAS mice treated with 2-HOBA. (FIG. 22A) The level of pH2AX in the gastric tissues of FVB/N INS-GAS mice±H. pylori±2-HOBA was assessed by immunostaining. The images are representative of 2 mice each for the uninfected and uninfected+3 mg/ml 2-HOBA groups, and 3 mice each for all the groups of H. pylori-infected mice. Scale bar, 50 μm. (FIG. 22B) The staining was quantified in a blinded manner by our GI pathologist. For each animal, the number of pH2AX-positive cells was counted in 5 high power fields (HPF; 400X), from the proximal corpus to the transitional mucosa of the corpus and antrum. P was calculated by one-way ANOVA and Tukey test. (FIG. 22C-FIG. 22D) The level of pH2AX in AGS cells±H. pylori±2-HOBA was assessed by immunofluorescence (FIG. 22C); green, pH2AX; blue, DAPI; turquoise, merged images. The percentage of cells with a positive staining for pH2AX in the nucleus was determined in a blinded manner from 3 different HPFs per condition (FIG. 22D). The data are derived from three independent experiments. Scale bar, 50μm.

### DESCRIPTION OF THE INVENTION

The present invention can be understood more readily by reference to the following detailed description of the invention and the Examples included therein.

Before the present compounds, compositions, articles, systems, devices, and/or methods are further disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “levuglandin scavenger” is a compound that prevents reactive carbonyls such as levuglandin from reacting with DNA and proteins. Without being bound by theory or mechanism, this may occur by reacting with the carbonyls to form covalent adducts, thus preventing them from forming adducts of DNA and proteins. Examples include covalent adducts on DNA bases and on lysines (Lys-LG) in histones.

One embodiment of the present invention is a method of treating gastrointestinal cancer, including colorectal cancer (CRC). Despite intensive efforts at colonoscopy-based screening, the disease burden of CRC remains vast; it has the third highest incidence and the second highest mortality rate of all cancers. While there has been great interest in chemoprevention strategies, e.g. aspirin2, COX2 inhibitors, difluoromethylornithine+sulindac4, there is no effective strategy that has impacted on clinical practice. Further, despite many studies of oncogene and tumor suppressor gene alterations, the exact molecular mechanisms by which malignant lesions occur in the epithelium remain unclear. There are high-risk populations, including patients with: 1) longstanding inflammatory bowel disease (IBD), and 2) history of high-risk adenomas or genetic abnormalities (e.g. familial adenomatous polyposis coli; FAP) that are strong candidates for chemoprevention.

Under conditions such as inflammation and/or accumulated mutations, epithelial cells and adjacent immune cells express genes encoding enzymes involved in carcinogenesis, such as prostaglandin-endoperoxide synthase (PGHS2; COX2), NADPH oxidases, and spermine oxidase. The products of these enzymes (prostaglandins, O2D, and H2O2 plus 3-aminopropanal) lead to formation of dicarbonyl electrophiles: levuglandins (LGs), malondialdehyde (MDA), 4-oxo-nonenal (4-O NE), and acrolein. Electrophiles may lead to immune dysfunction and neoplastic risk by forming adducts with proteins and DNA11-15. However, the role of these reactive aldehydes in colon carcinogenesis is previously unknown. The present inventors have discovered that the level of LG-protein adducts is increased in the colon tissues of i) human ulcerative colitis (UC), colitis-associated carcinogenesis (CAC), and CRC; ii) mice treated with azoxymethane-dextran sulfate sodium (AOM-DSS), a model of CAC; and iii) transgenic mice with a tamoxifen-inducible disruption of Apc using the colon-specific, caudal type homeobox 2 (CDX2) Cre (CDX2P-CreERT2;Apcff), which recapitulates sporadic/genetically-driven CRC. The present inventors have also discovered that compounds of the present invention, including 2-hydroxybenzylamine (2-HOBA) (salicylamine) and 5-ethyl-2-hydroxybenzylamine (EtHOBA) react with dicarbonyl electrophiles at a rate 3 orders of magnitude faster than lysine, thus preventing adduct formation with cellular macromolecules. 2-HOBA is a natural product derived from buckwheat seeds. It is not toxic or mutagenic and protects mice from oxidative damage in models of hypertension and Alzheimer's disease. A phase I clinical safety trial in humans has been completed at Vanderbilt University Medical Center (VUMC; NCT03176940)19, and other trials are ongoing. Additionally, the present inventors show that compounds of the present invention significantly reduce tumor development and dysplasia in the AOM-DSS model. The present inventors also show that mice treated with compounds of the present invention show increased colonic immune responses to tumors.

Cyclooxygenase-2 (COX-2) expression is associated with the development of many cancers, and the enzyme plays a key role in the progression of chronic gastrointestinal inflammation to cancer. Predictably, treatment with COX inhibitors decreases a person's total risk of cancer. Prevention studies as well as animal models suggest that increased COX-2 activity is both an early event in carcinogenesis, which contributes to the cellular transformation to malignancy, as well as a sustained event in some colorectal and lung cancers that can be associated with metastasis and poorer clinical prognosis. As predicted by these data, inhibiting COX-2 activity with non-steroidal anti-inflammatory drugs (NSAIDS) or COX-2-specific inhibitors over time reduces a person's total risk of colon, breast, lung, and prostate cancers.

Despite the promise of these drugs in cancer prevention, the gastrointestinal toxicity associated with long-term NSAID treatment and increased cardiovascular events associated with COX-2-specific inhibitors limit their clinical use. A better understanding of the specific downstream contributions of COX-2 to carcinogenesis could lead to new treatments that bypass these undesirable effects.

The product of COX-2, prostaglandin H2 (PGH2), is converted enzymatically into other prostaglandins, and indeed PGE₂ is a well-described promoter of carcinogenesis. However, depending on the animal model, deletion of microsomal PGE₂ synthase-1 can either prevent or accelerate tumorigenesis, indicating that the contribution of COX-2 to cancer, particularly to cellular transformation, is probably multifaceted.

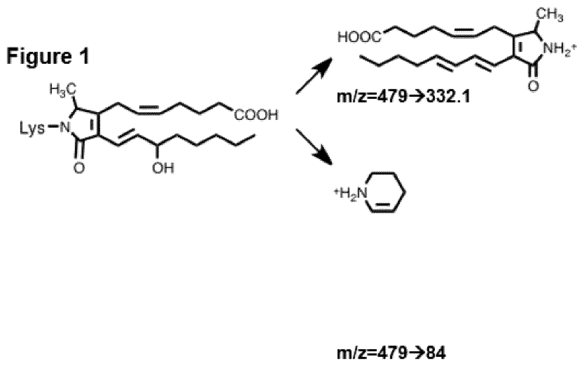

Besides enzymatic conversion, PGH2 also spontaneously rearranges in aqueous solution to form the highly reactive levuglandins, LGE₂ and LGD₂. The levuglandins (LGs) constitute about 20% of total PGH₂ rearrangement products. Newly formed LGs react almost immediately with free amino groups, such as those in lysine, which leads to stable covalent LG-protein adducts measureable by mass spectrometry (FIG. 1) or protein-protein crosslinks. Following COX-2 activity, LG adducts of protein form in cells and in tissues. Proteins rich in lysine are thus especially susceptible to adduction, and due to the perinuclear localization of COX-2 and a PGH₂ half-life measured in minutes, we speculated that it would be possible for PGH₂ to cross the nuclear envelope before rearranging to LGE₂, allowing formation of LG adduct on the lysine-rich histones.

The present inventors have discovered LG-histone adducts in multiple cancer cell lines as well as rat liver, with highest measurable amounts of adduct on the H4 histone. Adduct formation is dependent on COX-2 expression or activity. Lysines, with their short sidechain and positive charge, are critical for histone ionic interaction with DNA, and the inventors find that this interaction is decreased with the introduction of a hydrophobic negative charge from LG. Covalent histone modifications are a major method of controlling gene expression. Changes to lysyl modifications of histones are associated with human cancer. These findings link COX-2 induction with perturbation of normal DNA-histone interactions and provide a novel role for the enzyme in carcinogenesis.

Importantly, the inventors have found a small-molecule salicylamine derivative that scavenges LG to reduce adduct formation on histones without affecting COX-2 activity. In comparison with other scavengers, salicylamine, pyridoxamine and their other analogues, ethylsalicylamine was most potent in protecting histones from modification by levuglandins, indicating that it has the key property of being transported into the nucleus.

As stated above, Helicobacter pylori (Hp) induces an innate immune response in epithelial and myeloid cells that leads to inflammation-associated cancer. The inflammatory effector molecules, such as prostaglandins and reactive oxygen species, can generate the bifunctional electrophiles: levuglandins (LG), malondialdehyde, 4-oxo-nonenal, and acrolein. These molecules form covalent adducts on DNA bases and on lysines (Lys-LG) in histones, thus disrupting DNA/histone interactions and increasing risk for mutations. Embodiments of the present invention scavenge said electrophiles, including 5-ethylsalicylamine (EtSA), for example, which prevents adduct formation.

As discussed further in the Examples, the human gastric epithelial cell line AGS was infected with Hp PMSS1±10 mM EtSA. FVB/N insulin-gastrin (INS-GAS) mice and Mongolian gerbils were infected for 8 wk with Hp PMSS1 and 7.13, respectively; EtSA (0.75% in water) was given to animals beginning one wk post-infection. Lys-LG adducts, a marker of electrophile reactions, and EtSA were quantified by LC/ESI/MS/MS. Histology was analyzed on H&E staining. DNA damage was evaluated by pH2AX staining. Hp colonization was quantified by culture. Mucosal immune responses were determined by RNA profiling and by Luminex assay. Mice were imaged using PET/CT with ¹⁸F-FDG (inflammation marker) and ¹⁸F-NaF (tumor marker).

Lys-LG adduct levels were increased in AGS cells infected with Hp and inhibited by EtSA. Similarly, Lys-LG adducts were significantly increased by 2.3-fold in infected mice compared to uninfected animals (p<0.05), and were completely inhibited by EtSA, which was
found to be bioavailable in the stomach. There were decreases in frequency of intramucosal carcinoma (from 69% to 28%; p<0.05) and extent of dysplasia and carcinoma (p<0.05) in Hp-infected INS-GAS mice treated with EtSA. DNA damage was increased by 5.8-fold in infected mice and was decreased by 71% with EtSA treatment (p<0.05). Levels of Hp colonization, histologic gastritis, and tissue cytokines and chemokines were not affected by EtSA in INS-GAS mice. These data were confirmed by imaging: gastric uptake of the tumor marker ¹⁸F-NaF was induced by Hp and decreased by EtSA, while uptake of the inflammation marker ¹⁸F-FDG was increased in infected mice, but not modified by EtSA. Similarly, the frequency of gastric cancer development was reduced in gerbils treated with EtSA.

This shows that Hp-induced gastric carcinogenesis is inhibited by the electrophile scavenger EtSA, without affecting inflammation. Thus, without being bound by theory or mechanism, embodiments of the present invention act downstream of inflammatory reactions by removing electrophiles, which are a link between inflammation and the molecular alterations that lead to neoplastic transformation. The electrophile scavengers of the present invention serve as valuable chemopreventive agents.

### Compounds

It is understood that the following disclosed compounds can be employed in the disclosed methods of using or treating.

Examples of these compounds include, but are not limited to, compounds selected from the formula:

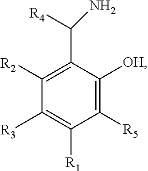

wherein:
R₁ is H, D, D₂, substituted or unsubstituted alkyl, substituted or unsubstituted alkoxy, carboxyl, substituted or unsubstituted aryl, or substituted or unsubstituted cycloalkyl;
R₂ is H, substituted or unsubstituted alkyl, substituted or unsubstituted alkoxy, alkyl-alkoxy-R₄;
R₃ is H, halogen, substituted or unsubstituted alkyl, substituted or unsubstituted alkoxy, hydroxyl, nitro;
R₄ H, D, D₂, substituted or unsubstituted alkyl, substituted or unsubstituted alkoxy, carboxyl, substituted or unsubstituted aryl, or substituted or unsubstituted cycloalkyl;
R₅ is H, substituted or unsubstituted alkyl, substituted or unsubstituted alkoxy;
or analogs thereof; and pharmaceutical salts thereof.

Without being bound by theory or mechanism, certain R₄ substituents are believed to impede metabolism by monoamine oxidase.

Examples of these compounds also include, compounds selected from the formula:

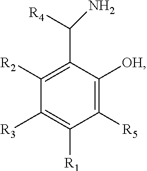

wherein:
R₁ is CH₃ or alkyl;
R₂ is H, substituted or unsubstituted alkyl, substituted or unsubstituted alkoxy, alkyl-alkoxy-R₄;
R₃ is H, halogen, substituted or unsubstituted alkyl, substituted or unsubstituted alkoxy, hydroxyl, nitro;
R₄ is a CH₃ or alkyl;
R₅ is H, substituted or unsubstituted alkyl, substituted or unsubstituted alkoxy;
or analogs thereof; and pharmaceutical salts thereof.

In other embodiments, R₂ is H, R₅ is H, and R₄ is CH_{3.}

Further examples of compounds or analogs of the present invention include compounds of the following formula:

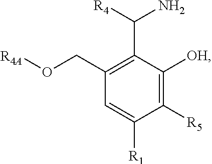

wherein R₁, R₄, and R₆ are defined above. R₄A is independent from R₄ and is H, D, substituted or unsubstituted alkyl, substituted or unsubstituted alkoxy, carboxyl, substituted or unsubstituted aryl, or substituted or unsubstituted cycloalkyl; or an analog thereof, and pharmaceutical salts thereof.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “alkoxy” group includes an alkyl group as defined above joined to an oxygen atom having preferably from 1 to 10 carbon atoms in a straight or branched chain, such as, for example, methoxy, ethoxy, propoxy, isopropoxy (1-methylethoxy), butoxy, tert-butoxy (1,1-dimethylethoxy), and the like.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The terms “amine” or “amino” as used herein are represented by a formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

Examples of compounds or analogs of the present invention include compounds of the following formula:

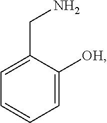

or an analog thereof, and pharmaceutical salts thereof.

Further examples of compounds or analogs of the present invention are 2-hydroxybenzylamine, methyl-2-hydroxybenzylamine, and ethyl-2-hydroxybenzylamine.

Further examples of compounds or analogs of the present invention include compounds of the following formula:

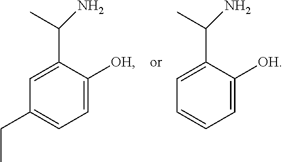

or an analog thereof, and pharmaceutical salts thereof.

Further examples of compounds or analogs of the present invention include compounds of the following formula:

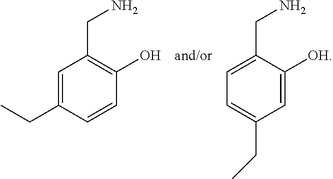

Further compounds or analogs may also be chosen from:

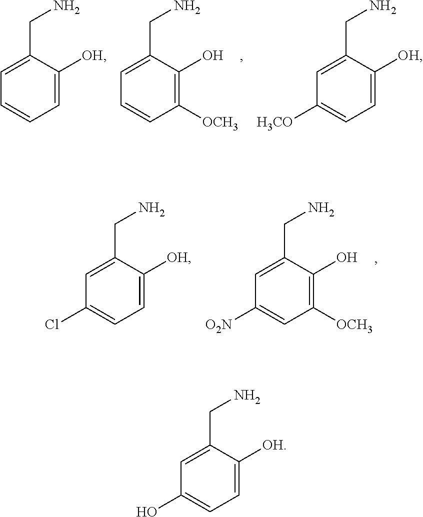

or an analog thereof.

The compounds may also be chosen from:

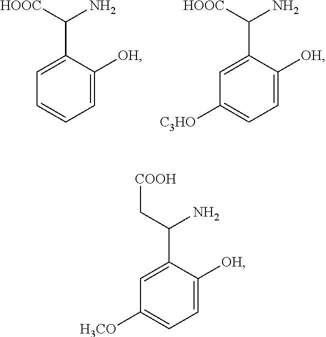

or an analog thereof.

The compounds of the present invention can also be chosen from:

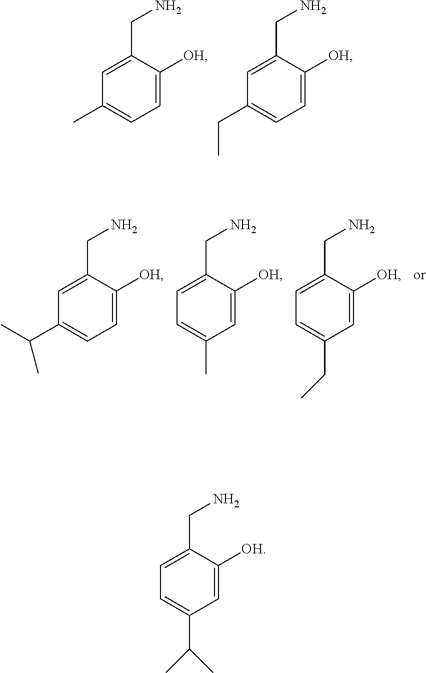

The compounds of the present invention can be administered as the sole active pharmaceutical agent, or can be used in combination with one or more other agents useful for treating or preventing various complications, such as, for example, lesions and cancer. When administered as a combination, the therapeutic agents can be formulated as separate compositions that are given at the same time or different times, or the therapeutic agents can be given as a single composition.

The compounds of the present invention may be made up in a solid form (including granules, powders or suppositories) or in a liquid form (e.g., solutions, suspensions, or emulsions). They may be applied in a variety of solutions and may be subjected to conventional pharmaceutical operations such as sterilization and/or may contain conventional adjuvants, such as preservatives, stabilizers, wetting agents, emulsifiers, buffers etc.

For administration, the compounds of the present invention are ordinarily combined with one or more adjuvants appropriate for the indicated route of administration. For example, they may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, they may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

In therapeutic applications, the compounds of the present invention may be administered to a patient in an amount sufficient to reduce or inhibit the desired indication. Amounts effective for this use depend on factors including, but not limited to, the route of administration, the stage and severity of the indication, the general state of health of the mammal, and the judgment of the prescribing physician. The compounds of the present invention are safe and effective over a wide dosage range. However, it will be understood that the amounts of compound actually administered will be determined by a physician, in the light of the above relevant circumstances.

The compounds of the present invention may be administered by any suitable route, including orally, parentally, by inhalation or rectally in dosage unit formulations containing conventional pharmaceutically acceptable carriers, adjuvants, and vehicles, including liposomes. The term parenteral as used herein includes, subcutaneous, intravenous, intraarterial, intramuscular, intrastemal, intratendinous, intraspinal, intracranial, intrathoracic, infusion techniques, intracavity, or intraperitoneally. In a preferred embodiment, ethylsalicylamine is administered orally or parentally.

Pharmaceutically acceptable acid addition salts of the compounds suitable for use in methods of the invention include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glutamine, etc. (see, e.g., Berge et al., J. Pharmaceutical Science, 66: 1-19 (1977).

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

### EXAMPLES

The following examples and discussion are to be construed as being exemplary of the present invention, and not intended to be limiting thereof.

### Example 1. Increased Electrophile Adduct Formation in Patients With CAC

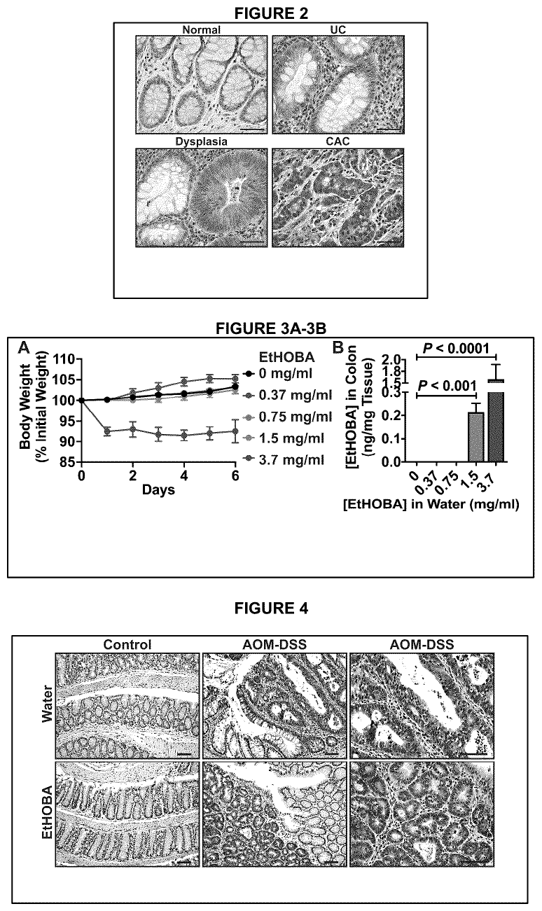

The present inventors performed immunohistochemistry (IHC) staining using the single chain antibody (Ab) D11, which specifically binds to L G-protein adducts, and a tissue microarray (TMA) from VUMC that contains colon tissues from patients with ulcerative colitis (UC), and associated precancerous and cancerous lesions. Histopathologic diagnoses were established from clinical case material from surgical resections and confirmed by H&E staining of the TMA. High levels of L G-protein adducts were present in active colitis, dysplasia, and carcinoma compared to normal patients (FIG. 2). On the TMA, 7/9 (78%) high grade dysplasia (HGD) cores and 21/23 (91%) CAC cores were scored positive for L G adducts. The strongest nuclear staining was observed in patients with CAC (FIG. 2), supporting the concept that electrophiles may thus affect histones and DNA.

### Example 2. Bioavailability of EtHOBA in the Colon

The present inventors analyzed the effect of various concentrations of EtHOBA and its bioavailability in control mice. Animals that were given 3.7 mg/ml EtHOBA lost weight on day 1, but did not lose additional weight and did not die (FIG. 3A). Mice treated with 0.37-1.5 mg/ml had no loss of body weight compared to untreated mice (FIG. 3A). After 6 days of treatment, EtHOBA concentrations in the colon were analyzed using liquid chromatography electrospray ionization tandem mass spectrometry (LC/ESI/MS/MS). The EtHOBA scavenger was readily detected in mice treated with 1.5 and 3.7 mg/ml (FIG. 3B). There was i) no macroscopic evidence of organ damage and ii) no change in colon weight/length in mice treated with 1.5 or 3.7 mg/ml EtHOBA (not shown).

### Example 3. Compounds of the Present Invention Reduce the Development of CAC

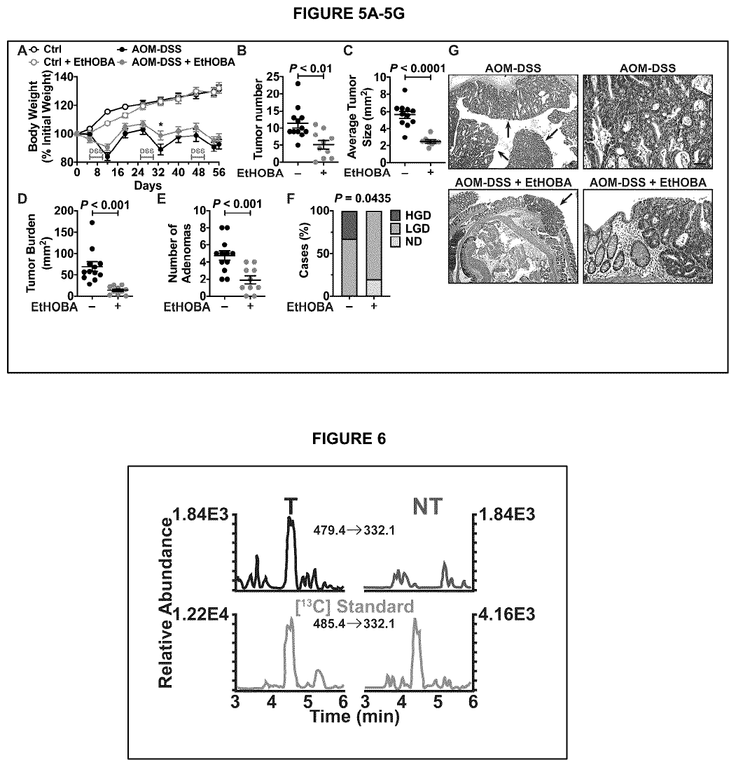

Animals receive an i.p. injection of the carcinogen AOM (12.5 mg/kg) followed by 3 cycles of 4% DSS, each for 4 days, over a total of 56-77 days. To directly determine the role of electrophiles in colon carcinogenesis, the present inventors tested the effect of EtHOBA in C57BL/6 mice in the AOM-DSS model. Mice were given 1.5 mg/ml EtHOBA one day after AOM and continued throughout the experiments, except during the DSS cycles, to avoid mixing. The present inventors assessed electrophiles in colons of mice treated with AOM-DSS, by IHC for electrophile protein adducts. There was markedly increased staining in mice with experimental CAC compared to control animals, predominantly in tumor areas (FIG. 4), including strong epithelial nuclear staining, indicating that electrophiles reach the nucleus and can affect histones and DNA. There was a marked attenuation of L G-protein adducts in mice treated with the EtHOBA electrophile scavenger (FIG. 4). There was no change in body weight in control mice treated for 56 days with EtHOBA (FIG. 5A), as with the acute treatment (FIG. 3A). At each cycle of DSS, mice had a decrease of body weight (FIG. 5A); there was less weight loss in animals given EtHOBA (FIG. 5A). Most importantly, there were significantly less tumors (FIGS. 5B, 5F), lower tumor size (FIG. 5C) and total tumor burden (FIG. 5D), and a reduced number of histologic adenomas (FIG. 5E) in the AOM-DSS group treated with EtHOBA. Histologic assessment further revealed that all the mice treated with AOM-DSS alone had either high grade dysplasia (HGD), or low-grade dysplasia (L GD; FIGS. 5F-G). In total, without the scavenger treatment, 25% of mice had HGD and the remaining 75% had L GD; in contrast, in those receiving EtHOBA, 20% had no dysplasia, none had HGD, and 80% had only L GD, a significant overall difference in diagnoses (FIGS. 5F-G). These data indicate that scavenging of electrophiles by EtHOBA can lead to reduction of inflammation-induced neoplastic transformation in the colon.

### Example 4. Increased Electrophile Adduct Formation in Patients With Colon Cancer

The present inventors analyzed the abundance of lysyl-L G adducts by LC/ESI/MS/MS11,12 in tumor and non-tumor tissues (from our collaborator, Dr. K. Washington) from patients with sporadic CRC. The level of lysyl-L G adducts in tumors was higher than in normal tissues (FIG. 6), supporting the concept that electrophiles and their adducts are generated in the human colonic mucosa and are reactive in tumors. This also demonstrates that lysyl-L G adducts can be measured from colon tissues using LC-MS, which is needed for this project.

### Example 5. Lysyl-LG Adducts Are Increased in Mice With a Model of Enetic/Sporadic Colon Cancer

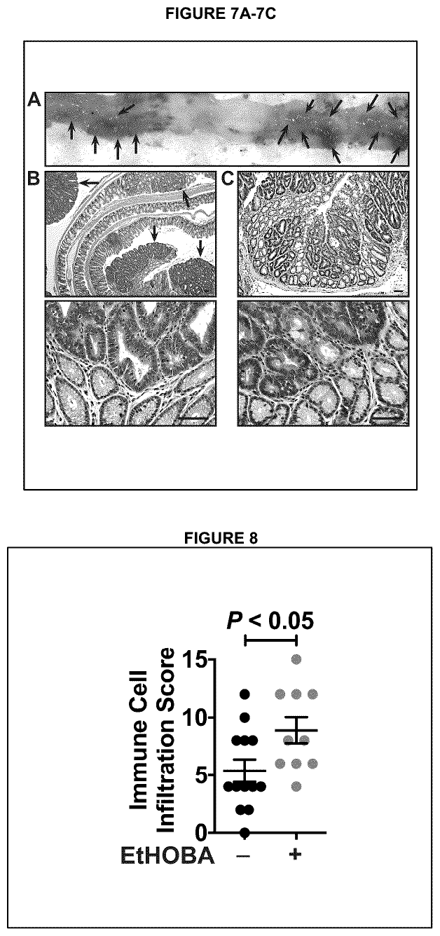

To mimic human CRC associated with APC loss, the present inventors used C57BL/6 CDX2P-CreER^{T2}; Apc^{fl/fl} mice. APC loss is a sentinel event in CRC, due to germline transmission as in FAP or its variants (e.g. attenuated FAP) with 100% penetrance. It is also a hallmark of sporadic CRC. This mouse model has been developed and optimized over the last decade. A major advance has been the use of the Apc double-floxed mice (Apc^{fl/fl}) mouse, which greatly increases the speed of tumor penetrance compared to the single flox (Apc^{fl/+}), combined with the tamoxifen inducible Cre, which allows for precise control and timing of the model. A single dose of 25 mg/kg i.p. of tamoxifen for these mice. These mice lose Apc gene function in the colon when treated with tamoxifen and exhibit tumors. The present inventors have found that mice show a 100% penetrance of tumors in male mice after tamoxifen, with many colon and rectal tumors seen (FIG. 7A). Histologically, the adenomatous tumors are large and readily detected on Swiss-rolls, with features of high-grade dysplasia (FIG. 7B). To verify the generation of electrophiles in this genetic/sporadic CRC model, the present inventors stained the colon of the tamoxifen-induced CDX2P-CreER^{T2}; Apc^{fl/fl} mice using the D₁₁ antibody. Dysplastic glands displayed marked staining for L G protein adducts compared to surrounding areas (FIG. 7C) and to control animals (not shown).

### Example 6. An Electrophile Scavenger Increases Immune Cell Infiltration in Mice With CAC

The present inventors used the tissues generated for FIG. 5 to study immune cell responses in AOM-DSS-treated mice±EtHOBA. It was discovered that more influx of immune cells in mice treated with EtHOBA compared to untreated mice (FIG. 8). EtHOBA had no effect on histology in control mice (not shown). Together with the data depicted in FIG. 5, results indicate that the electrophile scavenger both reduces the development of tumors in the AOM-DSS model and stimulates immune cell response. Because scavenging of electrophiles led to more immune cells in the colon, this suggests that electrophiles dampen mucosal immune responses.

### Example 7. The Colonic Antitumoral Immune Response is Increased by EtHOBA

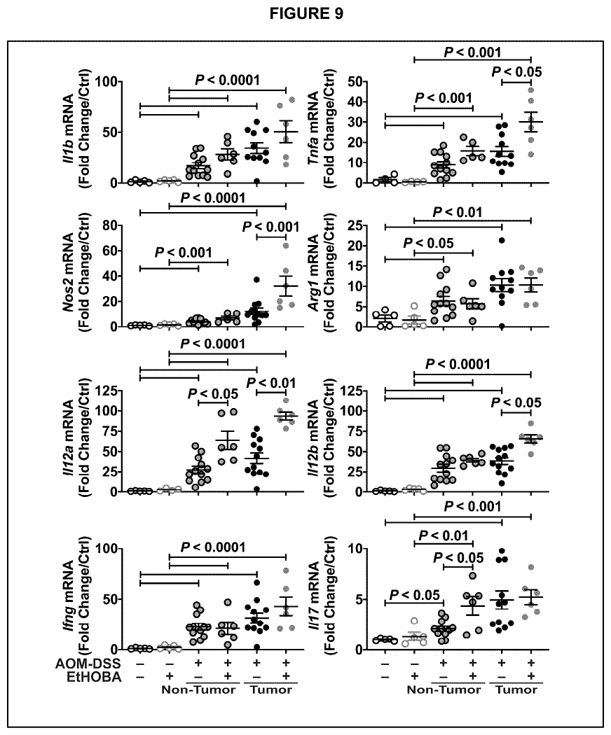

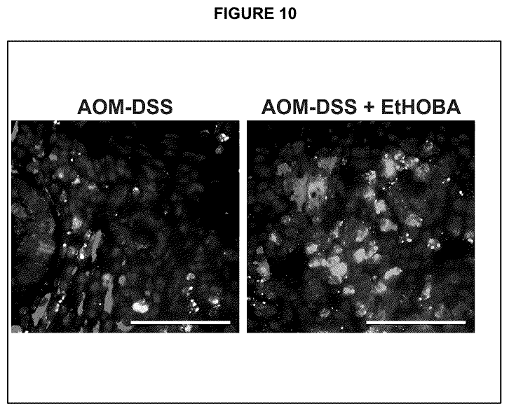

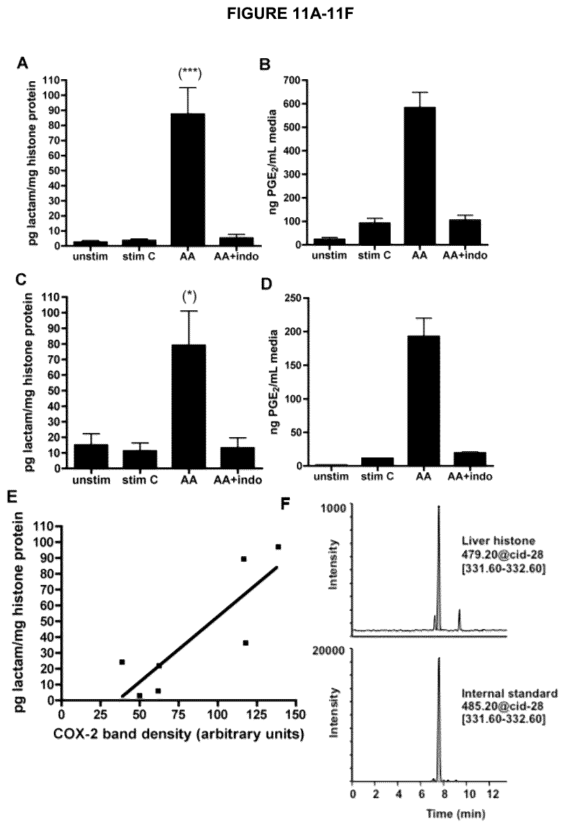

Using the tissues from the same mice as in FIG. 5, the present inventors analyzed the expression of key genes encoding mediators of the innate and adaptive immune response. The transcripts of the prototype i) M1 macrophage cytokines IL-1β (ll1b), TNF-α (Tnfa), IL-12A (ll12a), and IL-12B (1112b), and the enzyme NOS2 (Nos2); ii) M2 macrophage enzyme ARG1 (Arg1), iii) Th1 cytokine IFN-γ (lfng), and iv) Th17 cytokine IL-17 (ll17) were each significantly induced in the non-tumor and tumor tissues of AOM-DSS-treated mice compared to control animals (FIG. 11). Importantly, the overall pattern was that the genes encoding antitumoral effectors were expressed at a higher level in mice that were given compounds of the present invention. The following reached statistical significance for an increase with EtHOBA treatment: Tnfa (tumor), Nos2 (tumor), ll12a (non-tumor and tumor), ll12b (tumor), and ll17 (non-tumor) in this experiment (FIG. 9), which was limited by the reduced number of mice in the EtHOBA group with tumors. In contrast, the gene encoding the immunosuppressive myeloid mediator AR G1 was not affected by EtHOBA (FIG. 9). Using IF, we confirmed that mice treated with AOM-DSS mice that were given EtHOBA had increased CD68⁺NOS2⁺ (M1 macrophages) in colon tumors (FIG. 10).

These results suggest that the treatment of mice with experimental CAC with an electrophile scavenger exhibit more innate immune responses, including in the tumors, and potentially enhanced adaptive immune responses. Because compounds of the present invention reduce the number and progression of tumors in the colon (FIG. 5), one mechanism of action may be restoration of antitumoral immunity. The present inventors previously reported that mice with myeloid-specific deletion of ornithine decarboxylase, the rate-limiting enzyme for polyamine synthesis, developed less tumors in the AOM-DSS model—a finding that was strongly associated with enhanced immune cell infiltration in the non-tumor area and increased M1 cytokines and NOS233. These results indicated that polyamines impair antitumoral

### Example 8. COX-2 Activity and Histone Adduction

### Experimental Procedures

### Materials

All reagents and chemicals were purchased from Sigma-Aldrich (St Louis, MO) unless otherwise noted. Methanol and acetonitrile were from Fisher Scientific (Pittsburgh, PA) and were HPLC grade or higher. [14C]-arachidonic acid (AA) was obtained from Perkin-Elmer Life Sciences (Boston, MA). The following reactive dicarbonyl scavenger molecules were synthesized by V. Amarnath as previously described: pentylpyridoxamine (PPM), 3-methoxysalicylamine (3-MoSA), and 5-ethylsalicylamine (EtSA).

### Treatment of Cells

To stimulate COX-2 expression, A549 or RAW264.7 cells were treated overnight with 5 ng/ml IL-1ß (A549) or 10 μg/mL LPS and 10 U/mL IFNγ (RAW264.7) in serum-free medium. When indicated, cells were pretreated with indomethacin, aldehyde scavengers (glucosamine, 3-MoSA, PPM or EtSA), or vehicle (ethanol for indomethacin, H₂O for scavengers) for 45 min, and then given 20 μM arachidonic acid (AA) or DMSO vehicle for 1 h before lysing.

### Histone Extraction

Cultured cells were lysed in hypotonic buffer (10 mM Tris/10 mM NaCl/3 mM MgCl₂) containing 1 mM pyridoxamine and 100 μM indomethacin to prevent the artifactual formation of LGE₂ during the lytic process. After letting cells swell on ice, membranes were disrupted by addition of Triton X-100 (0.5% final concentration) and vortexing. Nuclei were isolated by centrifugation for 10 min at 1000×g, and the resulting pellet washed with PBS. Histones were extracted in 0.4 N H₂SO₄, precipitated with trichloracetic acid, and washed with acetone. With this method, histones are the predominant proteins and contaminating nuclear proteins are reduced (FIG. 3A). Histones were resolubilized in dilute NaOH, and PH neutralized with HCl. Protein concentration was determined using the method of Bradford. For tissue, a portion of frozen liver was homogenized in buffer containing 40 mM sodium citrate, and 1% Triton X-100 with 3 mM Trolox and 100 μM indomethacin. The supernatant was separated from settled debris, and nuclei were pelleted at 500×g and washed. Nuclear pellets were frequently transferred to fresh tubes to avoid contamination by floating lipid debris; any remaining was removed with a cotton swab. Histones were extracted as above. All centrifugation steps were carried out at 4° C.

### Sample Preparation and Mass Spectrometry

Histone samples were prepared for mass spectrometry by addition of ammonium bicarbonate to 5 mM final concentration before digesting to single amino acids by protease step-digestion as previously described. In the case of immunoblots, proteins were digested directly off the nitrocellulose through incubation of the nitrocellulose strip in 30 μg/mL Pronase in ammonium bicarbonate buffer, and later addition of aminopeptidase. All samples were centrifuged at 2000×g for 10 min. after final digestion to remove precipitate, spiked with 0.2 ng ¹³C-lysyl-lactam internal standard, and purified on prepared tC18 cartridges (Waters Corp., Milford, MA). Samples on tC18 cartridges were washed with water, then 30% methanol, before being eluted in 80% methanol and concentrated by evaporation. Samples were evaluated by electrospray ionization (ESI) LC/MS/MS on a ThermoFisher TSQ Quantum triple quadrapole mass spectrometer in positive ion mode and quantitated by isotopic dilution as previously described, with the exception of a reduced flow rate of 0.1 mL/min.

### Measurement of PGE₂

A sample of cellular media was taken just prior to lysis and centrifuged to remove any cellular debris. For PGE₂ analysis, samples were spiked with 2 ng of [²H₇] PGE₂ as an internal standard. Prostaglandins were isolated and derivatized for analysis by GC/MS, operating in negative ion chemical ionization (NICI) mode and monitoring selected ions as previously described . For the [²H₄] PGE₂ internal standard, m/z=528. To account for the deuterium-protium exchange at the position C12 of [²H₇] PGE₂, the summation of the signals obtained at m/z=530, m/z=531 and m/z=532 was performed.

### Autoradiography

A549 or RAW264.7 cells were treated overnight to stimulate COX-2 expression and given 20 μM ¹⁴C-AA (116 μCi) for 1 h. Histones were isolated as described above and separated on 4-12% SDS-PAGE gels (Life Technologies), which were stained with Coomassie and exposed to film for 2 weeks.

### Salt Extraction

60-80% confluent A549 cells were stimulated and treated with AA±500 μM EtSA before scraping cells in lysis media for nuclear isolation as above. After addition of Tritox X-100 and vortexing, 1.5 mL of each sample was aliquoted into an eppendorf and centrifuged to separate nuclei. Pellets were washed with PBS and all buffer removed. Nuclear pellets were resuspended in extraction buffer containing 0.6M, 0.9M, 1.2M or 1.5M NaCl (with 10 mM Tris, pH 7.5, 3 mM MgCl2, 0.5% NP-40, and protease inhibitor cocktail) and incubated 10 min on ice. Following this extraction period, the nuclei were centrifuged at 16,000×g to obtain the soluble fraction. This was sonicated, denatured at 95° ° C., and analyzed by SDS-PAGE and Western blotting, using Ponceau stain to visualize proteins and confirm equal loading, and anti-H4 antibody (Abcam, Cambridge, MA).

### Statistical Analyses

All data were analyzed using Prism software (GraphPad, La Jolla, CA). Data are expressed as means±SE, and statistical significance was determined using one-way ANOVA followed by Tukey's post-test or Dunnett's multiple comparisons post-test, when appropriate. A p value<0.05 was considered significant.

### Results

### Levuglandins Form Adducts on Histones in Cultured Cell and Whole Tissue

With mass spectrometry, the present inventors identified LG-lysyl adducts on histones in RAW264.7 macrophages (FIG. 11A) as well as A549 cultured lung epithelial cells (FIG. 11C). COX-2 is upregulated in these cells upon cytokine stimulation, and addition of exogenous AA leads to formation of LG-histone adducts. Formation of these adducts is blocked with indomethacin, further indicating a COX-dependent mechanism (FIG. 11A and 11C). Very few LG-histone adducts are formed in these cell lines without addition of exogenous AA, and PGE₂ analysis of cell media from each group indicates there is comparatively little endogenous AA mobilized following induction of COX-2 (FIG. 11B and 11D). Although few adducts are formed at basal levels in our cell lines, we find the LG-lysyl adduct in rat liver histones (FIG. 11E), where levels correlate with COX-2 expression, demonstrating COX-2-dependent adduct formation under physiological conditions.

### LG-Histone Adducts are Restricted to Specific Histone Isoforms

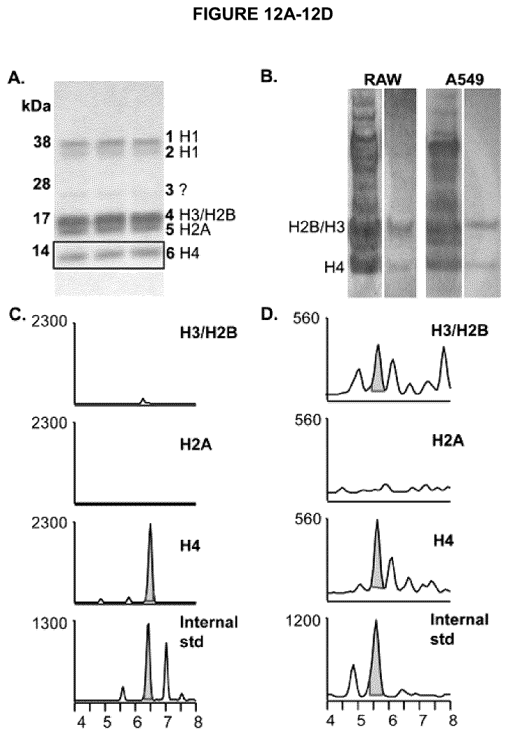

Using 0.4N H₂SO₄ to extract histones results in a relatively pure preparation, with histones corresponding to known molecular weights (FIG. 12A). Incubation of ¹⁴C-AA with stimulated A549 or RAW264.7 cells led to a ¹⁴C-containing band in the histone preparation that corresponded with H4 and H3/H2B, despite the fact that other histones are represented in equal or greater quantity (FIG. 12B). The present inventors treated stimulated RAW264.7 macrophages or A549 lung carcinoma cells with 20 μM AA for 1h and directly digested and analyzed the SDS-PAGE bands as labeled in FIG. 12A. The H4 band yielded a predominant peak corresponding with the internal LG-lysyl standard, while lower or no signal was seen in the H3/H2B band and no corresponding peaks were seen in other bands (FIG. 12C and 12D, data not shown). Thus, there is consistent evidence for formation of an LG-lysine lactam adduct on H4. The radiolabeled AA product adducted to H3/H2B probably includes structures in addition to the LG-lysine lactam. These results, from two separate cell lines, suggest that there is specificity in the reaction of LG with histones.

### The Scavenger 5-Ethylsalicylamine (EtSA) Reduces LG-Histone Adduct Formation Without Affecting COX-2 Activity

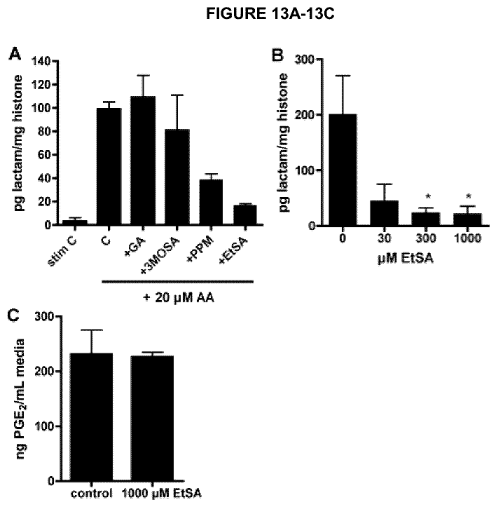

As demonstrated herein, in RAW264.7 cells, 5-ethylsalicylamine (EtSA) most effectively blocked adduct formation. Pentylpyridoxamine partially inhibited histone adduct formation, but was much less potent than 5-ethylsalicylamine (FIG. 13A). EtSA also inhibited LG-histone adduct formation in stimulated, AA-treated A549 cells, without affecting PGE₂ production at the highest concentration tested (FIG. 13B and C).

### Formation of LG-Lysyl Adduct on Histone H4 Decreases DNA-Histone Interaction

To examine the functional effect of LG-histone adduction, we performed salt fractionation of A549 nuclei to determine histone solubility. In this assay, loosely-bound histone is released at lower salt concentrations than tightly-bound proteins. The present inventors discovered that in stimulated, AA-treated A549 cells, histone H4 was eluted at lower salt concentrations than in stimulated control cells; this was reversed after treatment with the scavenger EtSA (FIG. 14).

### Discussion

As indicated herein, the present inventors established that COX-2 catalysis can cause changes in DNA-histone interactions through formation of LG-histone adducts, suggests a new hypothesis for the contribution of COX-2 to the etiology of cancer. Oxidative damage is known to cause N6-formylation of H1 histone, and epigenetic modification affecting COX-2 transcription is well-described, but the LG-lysyl histone adduct we describe here is an entirely novel finding that links inflammation and COX-2 activation with histone modification.

The present inventors discovered COX-2 dependent formation of LG-histone adducts in cells and tissues. Whereas COX-2 blockade by treatment with indomethacin decreases LG-histone adduct formation in A549 or RAW264.7 cells, this method of antagonism cannot separate the myriad effects of other COX-2 products from the effects of the LG-histone adducts. The present inventors screened a number of small molecule levuglandin scavenger molecules for their ability to decrease LG-histone adduction. LG will react with these molecules three orders of magnitude faster than lysine, and we have previously shown that scavenger treatment decreases total cellular levels of LG-lysine adducts without affecting PGE₂ production. These scavengers are orally bioavailable, and able to decrease total LG-protein adduct levels when given to mice in drinking water. In future studies, aside from allowing investigation of LG-protein modification independent of COX activity, use of these scavengers may bypass the cardiovascular and gastric side effects seen with COX inhibitors.

Interestingly, not all histones are targeted, but cellular LG adducts seem to preferentially form on the H4 and, to a lesser extent, on H3/H2B. Whether this specificity is a reflection of histone availability in the nucleosome, accessibility of lysine residues, or a more favorable microenvironment for adduct formation remains to be shown. Incubation with [¹⁴C]-AA in cells led to a stronger autoradiographic band at H3/H2B compared to H4, while LC-MS analysis indicated that H4 was the major form adducted by LGs. This discrepancy can be explained by several mechanisms. Protein-associated radioactivity would come from any product derived from [¹⁴C] AA, including LGs but also PGJ₂/PGA₂ cyclopentenone or arachidonate ester adducts. In addition, the occurrence of LG-lysyl adducts is almost certainly underreported with our current approach. Our internal standard and mass spectroscopy method are specific for detection of a single LG-lysyl adduct with an m/z equivalent to the lactam structure, but the initial Schiff base intermediate of LG-lysyl adducts can oxidize to form other structure such as hydroxylactam or intraprotein or protein-DNA crosslinks, which would go undetected in our method. For these reasons, the autoradiograms can not be quantitatively compared with the LC/ESI/MS/MS results as they do not measure the same molecular structures.

H4, along with H2A, H2B, and H3 histones, comprise the histone octamer around which DNA is “packaged” into nucleosomes. The interaction of histone N-terminal tails with DNA is critical to DNA compaction and organization, and is dependent on the numerous positively charged lysine and arginine residues present; a mesoscopic model demonstrates that H4 tails are the most important in mediating internucleosomal interactions. A single lysyl acetylation on K16 of H4 modulates chromatin compaction and interaction of numerous chromatin-associated proteins; constitutive acetylation of this residue confers a folding defect comparable to deletion of the entire H4 tail. After LG-histone adduct formation we do find disruption of histone-DNA binding, resulting in increased DNA extraction in a salt solution. This decreased histone-DNA interaction may increase DNA transcriptional access to previously silent oncogenes, and contribute to the development of cancer.

The complex patterns of lysyl acetylation and methylation comprise a “histone code” that regulates chromatin access and transcription; it is plausible that irreversible adduction of lysyl residues could disrupt this code, or directly alter the access of DNA-interacting proteins. Changes in histone modifications are known to result in altered DNA methylation, deregulation of oncogenes, genomic instability, impaired DNA repair, and defects in cell cycle checkpoints. Changes in lysyl modifications of H4 in particular are a common hallmark of human cancers, and are associated with a global loss of DNA methylation. Further elucidation of the effects of LG-histone adduction on histone modification, DNA-histone interactions, and transcription should increase our understanding of the molecular mechanisms whereby COX-2 contributes to cancer development and progression.

### Example 9. Inhibition of Formation of Levuglandin Adducts in an in Vivo Animal Model in Which Intramucosal Gastric Carcinoma Develops Following Infection With H. pylori

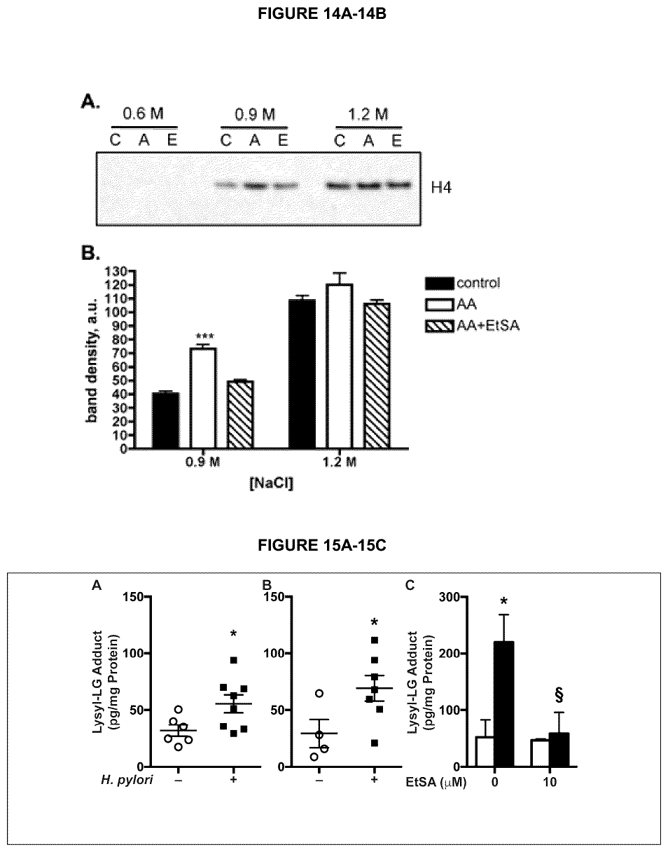

To demonstrate that bifunctional electrophiles are generated in vivo during carcinogenesis, the present inventors analyzed the concentration of lysyl-Levuglandin (lysyl-LG) adducts using liquid chromatography electrospray ionization tandem mass spectrometry (LC/ESI/MS/MS), in the whole gastric tissues of C57BL/6 mice (see FIG. 15A) and INS-GAS mice (FIG. 15B) infected by H. pylori. In both types of mice, lysyl-LG adduct levels were significantly increased in infected mice compared to uninfected animals, demonstrating that electrophiles are locally generated during the infection. To further determine whether ethylsalicylamine (EtSA) prevents the formation of bifunctional electrophiles during H. pylori infection, the mice were treated or not with EtSA 30 min prior to the infection. Our results show that lysyl-LG adduct concentration was significantly increased in H. pylori-infected AGS cells when compared to uninfected cells and completely attenuated by 10 μM EtSA (FIG. 15C).

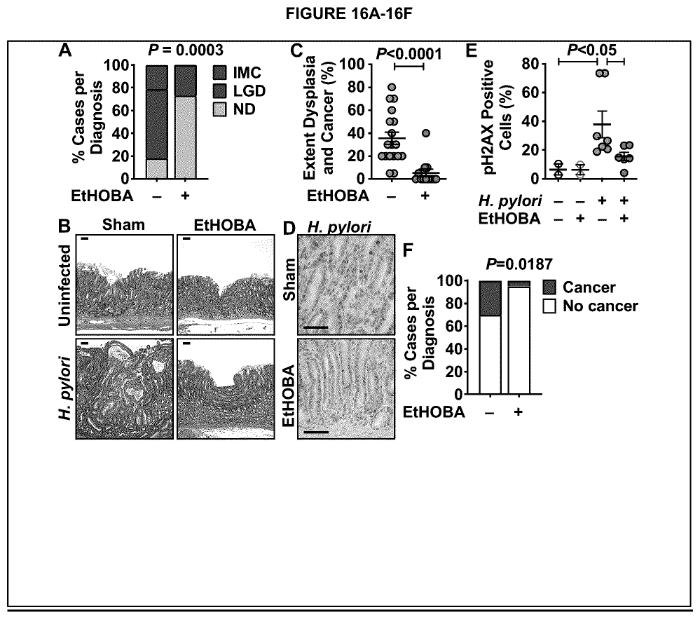

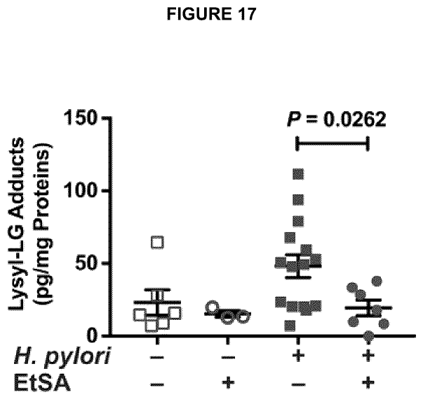

The effect of a scavenger of levuglandins and other dicarbonyls was investigated in transgenic INS-GAS mice, which have high circulating gastrin levels and develop atrophic gastritis, and dysplasia and carcinoma with H. pylori infection. Mice were infected with H. pylori, and then EtSA was administered in drinking water, starting 7 days after infection. After 8 weeks, EtSA levels were measured in the gastric tissues of uninfected and infected mice receiving this compound (FIG. 16A). There were significant decreases in development of dysplasia (from 37.5% to 14.3%) and carcinoma (from 37.5% to 0%) in H. pylori-infected INS-GAS mice treated with EtSA (FIG. 16B). Extent of dysplasia and carcinoma as a percentage of the gastric mucosa was also significantly attenuated by EtSA (FIG. 16C). FIG. 16D depicts hematoxylin and eosin (H&E) staining of the gastric mucosa of infected INS-GAS mice showing intramucosal carcinoma in an infected mouse, characterized by irregular and angulated glands with infiltration of the lamina propria by tumor cells and desmoplastic stroma; in contrast, infected mice treated with EtSA showed low grade dysplasia characterized by irregular proliferation of glands that do not infiltrate the stroma. There was no dysplasia or carcinoma in uninfected mice (FIG. 16D). It should be noted that gastric colonization by H. pylori was not reduced by EtSA treatment (data not shown), demonstrating that the protective effect of EtSA was unlikely due to an effect of EtSA on bacterial killing.

In summary, it is well established that in humans, H. pylori infection initiates an inflammatory process that leads to gastric cancer. The results of these investigations show that compounds of the present invention are useful for preventing the development of H. pylori-induced gastric cancer in humans.

### Example 10—2-HOBA and Stomach Cancer

### 2. Materials and Methods

### 2.1. Synthesis of 2-HOBA

2-HOBA (as the acetate salt, CAS 1206675-01-5) was obtained from TSI (China) Co., Ltd. (Shanghai, China). A commercial production lot was used (Lot SAA20200727). The purity was of the commercial lot was verified was HPLC to be >99%. Microbial and analytical tests were within all specification limits.

### 2.2. H. pylori

The cagA+ H. pylori strain PMSSI was used. Bacteria were maintained on Tryptic Soy agar plates containing 10% sheep blood. For infection, bacteria were grown in Brucella broth containing 10% FBS overnight, then diluted to an A600 nm of 0.1 in the same fresh medium, grown, harvested at the exponential phase, and resuspended into Brucella broth.

### 2.3. Mice, Infection, and Treatment With 2-HOBA

FVB/N INS-GAS mice (8-10 weeks-old) were infected by oral gavage with 109 H. pylori PMSS1 in 0.2 ml Brucella broth, two times every 2 days. Control mice were gavaged with broth only. Mice were then fed ad libitum with the AIN-76A diet (Bio-Serv) during the time of infection. Animals were treated with 1.45 or 4.35 mg/ml 2-HOBA-acetate, which corresponds to 1.0 and 3.0 mg/ml 2-HOBA, respectively, in the drinking water, beginning 7 days after the first infection. 2-HOBA supplementation was maintained until the end of the experiments and the solutions were changed every 3-4 days. Mice were sacrificed 56 days post-inoculation with H. pylori. Stomachs were harvested and analyzed as described. The number of H. pylori in each stomach was determined by counting the colony forming units (CFUs) after plating serial dilutions of ground gastric tissues.

Animals were used under protocol M1800038 approved by the Institutional Animal Care and Use Committee at Vanderbilt University, and the Vanderbilt University Institutional Biosafety Committee. Procedures were performed in accordance with institutional policies, AAALAC guidelines, the AVMA Guidelines on Euthanasia, NIH regulations (Guide for the Care and Use of Laboratory Animals), and the United States Animal Welfare Act (1966).

### 2.4. Gastric Epithelial Cells

The human gastric epithelial cell line AGS (ATCC #CRL-1739) was used, maintained in DMEM with 10% FBS, sodium pyruvate, Hepes, and penicillin-streptomycin. Cells (2X105) were plated in 8-well Lab-Tek chamber slides (Nunc), treated with 100 μM 2-HOBA for 2 hours, and then infected with H. pylori PMSS1 for 24 hours at a multiplicity of infection of 100 without antibiotics.

### 2.5. Histopathology

Longitudinal stomach segments were fixed in 10% neutral buffered formalin and stained with hematoxylin and eosin (H&E). Histologic assessments were then performed by a gastrointestinal pathologist (M.B.P.) in a blinded manner. Acute and chronic inflammation of the antrum and the corpus regions of the stomach (0-3 for each) were determined, leading to a final 0-12 score. Dysplasia and adenocarcinoma were diagnosed as described.

### 2.6. Measurement of 2-HOBA

Concentrations of 2-HOBA were determined in gastric tissues using a method that we previously described, with a few modifications. [2H4]-2-HOBA was used as an internal standard and was added (5 ng/μl) to all standards, quality control samples, and tissue samples. Standard and quality control samples of 1 mg/ml 2-HOBA were prepared in water. Seven standard curve samples (10, 20, 100, 200, 1000, 2000, and 5000 ng/ml) were prepared with stripped human serum (GoldenWest Diagnostic, Temecula, CA), NY). In addition, three quality control samples (15, 300, and 3000 ng/ml) were prepared. Tissue samples were homogenized with the internal standard and 200 μl of ice-cold PBS, vortexed, and centrifuged at 12,000×g for 10 min at 4 ° C. The supernatants were transferred to 13×100 glass culture tubes and then 400 μL of acetonitrile was added to samples, standards, and controls. The tubes were then vortexed for 2 min and then centrifuged for 5 min at 4° C. The supernatants were applied to a Solo HRP SPE column and the 2-HOBA was eluted with a 65/35 methanol/water solution and dried with a vacuum drier. The samples were reconstituted with 40 μl of LCMS-grade acetonitrile containing 0.2% formic acid and 40 μl of LCMS-grade water containing 0.2% formic acid and 0.1% ammonium formate. Liquid chromatography tandem mass spectrometry analysis of 2-HOBA was performed with Agilent 1290 pumps, autosampler, column oven, and degasser (Santa Clara, CA) (column: Agilent SB-C18 RRHD 1.8μm 2.1×100 mm) coupled with an Agilent 6460 mass spectrometer with ESI ion source in positive mode (Santa Clara, CA). The column temperature was set to 30 ° C. and the flow rate was 0.3 ml/min. A gradient of 50-70% B from 0 to 3.0 min was established by using a mobile phase A of 0.2% formic acid and 0.1% ammonium formate in water and mobile phase B of 0.2% formic acid in acetonitrile. Agilent MassHunter® software (Quant) was used to integrate 2-HOBA and [2H4]-2-HOBA and quantitate 2-HOBA in tissues.

### 2.7. Immunostaining

Gastric tissues sections were incubated at room temperature with 3% hydrogen peroxide in PBS to block endogenous peroxidase and blocked for 1 h in a solution of 5% human/mouse serum and 5% BSA in PBS. Slides were then incubated with an anti-phosphoserine 139 of H2A histone family member X (pH2AX) polyclonal antibody (1:200; Novus) overnight at 4° C. followed by 30 min at room temperature with the EnVision+, HRP (Dako). Visualization was performed using 3,3′-diaminobenzidine, and tissues were counterstained by hematoxylin. The number of positive cells were determined in a blinded manner by our GI pathologist (M.B.P.).

AGS cells were fixed in 3.7% paraformaldehyde, washed with PBS, and blocked with Universal Protein Block (Dako) for 40 minutes at room temperature. Cells were then incubated with the anti-pH2AX polyclonal antibody (1:200; Novus) overnight at 4° C. followed by 1 hour at room temperature with the Donkey anti-Rabbit IgG (H+L) Highly Cross-Adsorbed Secondary Antibody, Alexa Fluor Plus 488 (1:600; Invitrogen). Slides were mounted with VECTASHIELD HardSet™ Antifade Mounting Medium with DAPI (Fisher Scientific) and confocal images were acquired using the Cytation C10 Confocal Imaging Reader (BioTek).

### 2.7. Analysis of mRNA Levels

Total RNA was isolated using the RNeasy Mini kit (Qiagen). Reverse transcription was performed using Superscript II Reverse Transcriptase and Oligo dT (Invitrogen). mRNAs were amplified by real-time PCR using the PowerUp SYBR Green Master Mix (Thermo Fisher Scientific) and the primers listed in Supplementary Table S1.

### 2.8. Statistics

The in vivo data are derived from two independent experiments, with the results combined. Figures and statistics were generated by Prism 9.4.1. Dot plots and bar graphs represent the mean±SEM. Outliers were identified using the ROUT test (Q=5%) and removed from the analysis. Data that were not normally distributed according to the D'Agostino & Pearson normality test were log or square root transformed. Student's t test was used to determine significant differences between two groups, whereas analysis of multiple groups was performed using ANOVA with the Tukey test or the Dunnett's test. These tests were two-sided. Contingency analyses were performed by Chi-square test. For correlation, simple linear regression was used to determine the r value and P was calculated using the one-tailed Pearson test.

### 3. Results

### 3.1. 2-HOBA is Bioavailable in the Stomach

INS-GAS mice, infected or not with H. pylori, were treated with 1 mg/ml or 3 mg/ml 2-HOBA in the drinking water, according to the experimental design depicted in FIG. 18A. At the end of the experiment, we first determined the concentration of 2-HOBA by LC-MS/MS in the gastric tissue. We did not detect this compound in the stomach of uninfected or infected mice that were not treated with 2-HOBA (FIG. 18B). In contrast, 2-HOBA was found in the gastric tissues of animals that were given this scavenger drug, infected or not with H. pylori. In infected mice, we did not observe a significant difference in the levels of gastric 2-HOBA between INS-GAS mice treated with 1 mg/ml or 3 mg/ml 2-HOBA (FIG. 18B). But interestingly, the concentration was significantly decreased in H. pylori-infected mice receiving 3 mg/ml 2-HOBA compared to control animals treated with 3 mg/ml 2-HOBA.

These data demonstrate that a per os treatment with 2-HOBA is efficient to increase its concentration in the stomach and that 2-HOBA appears to be metabolized in the gastric tissues of H. pylori-infected mice.

### 3.2. 2-HOBA Dampens H. pylori Pathogenesis

We next assessed the effect of 2-HOBA on gastric colonization, inflammation, and carcinogenesis in INS-GAS mice, which develop accelerated gastric dysplasia and intramucosal carcinoma (IMC) after H. pylori infection. H. pylori colonization burden (FIG. 19A) was not affected by 2-HOBA treatment. As shown in the H&E staining (FIG. 19B), the gastric tissues of mice infected with H. pylori exhibited marked mucosal hyperplasia and infiltration of immune cells, both of which were less detected in mice given 2-HOBA. In addition, areas of stromal reaction and neoplastic cells infiltrating the lamina propria, which are features of IMC, were observed in infected mice, whereas low grade dysplasia (LGD) with irregular and angulated glands was found in infected animals that were given 2-HOBA (FIG. 19B). When the infiltration of immune cells was scored, we determined that gastritis in H. pylori-infected mice was significantly reduced by both concentrations of 2-HOBA (FIG. 19C). There were also significant decreases in the development of LGD and IMC in H. pylori-infected INS-GAS mice treated with the electrophile scavenger compared to infected animals not receiving the 2-HOBA compound (FIG. 19D); in mice receiving the higher dose of 3 mg/ml, there were no cases of IMC. Similarly, the extent of dysplasia and carcinoma was significantly attenuated by 2-HOBA (FIG. 19E). Lastly, 2-HOBA had no effect on the gastric tissues of uninfected mice (FIGS. 19B, 19D, 19E). We then tested the hypothesis that 2-HOBA concentration in the stomach would be protective against H. pylori-induced carcinogenesis and demonstrated that gastric 2-HOBA level was inversely correlated with the extent of dysplasia and cancer (FIG. 20).

Taken together, these data indicate that the electrophile scavenger 2-HOBA reduces H. pylori-induced gastritis and is a potent inhibitor of gastric carcinogenesis, without affecting colonization.

### 3.3. Reduction of Inflammation by 2-HOBA

Since we observed a reduction of gastritis in H. pylori-infected mice, we assessed the expression of the genes encoding for markers of inflammation. The levels of the transcripts encoding for the innate effectors IL-1β, TNF-α, and NOS2, the chemokine CXCL1, the Th17 cytokine IL-17, and the prototype Th1 cytokine IFN-γ were significantly decreased in infected mice that were given 2-HOBA compared to infected animals not receiving this agent (FIG. 21). The rate of inhibition was similar when INS-GAS mice were treated with 1 or 3 mg/ml 2-HOBA (FIG. 21). Again, 2-HOBA had no significant effect on gene expression in uninfected mice (FIG. 21).

### 3.4. 2-HOBA Reduces H. pylori-Induced DNA Damage

Dicarbonyl electrophiles can inflict damage to DNA by forming adducts on DNA and histones. Using immunohistochemistry, we found that the level of pH2AX, a reliable marker of DNA damage, was increased in the nuclei of gastric epithelial cells in INS-GAS mice infected with H. pylori compared to uninfected animals (FIG. 22A). The immunostaining was markedly reduced in infected mice that were given 3 mg/ml 2-HOBA (FIG. 22A). Then the staining on multiple animals was quantified and confirmed that infection was associated with enhancement of pH2AX-positive cells, and thus with DNA damage (FIG. 22B). The present inventors observed that the treatment with 3 mg/ml 2-HOBA resulted in a significant reduction of pH2AX-positive cells in uninfected and infected INS-GAS mice (FIG. 22B). A slight reduction of pH2AX staining, which did not reach statistical significance, was also observed in H. pylori-infected INS-GAS mice treated with the lower dose of 1 mg/ml of 2-HOBA (FIG. 22B).

Further, the present inventors found by immunofluorescence (FIG. 22C) and quantification (FIG. 22D) that H. pylori directly induced DNA damage in the gastric epithelial cell line AGS. When the infected cells were pre-treated with 2-HOBA, the present inventors observed a marked and significant reduction of pH2AX+ nuclei (FIGS. 22C-D).

### 4. Discussion

Because dicarbonyl electrophiles derived from lipid oxidation can form covalent adducts with macromolecules such as histones or DNA they represent ideal candidates to be targeted to dampen inflammation-mediated carcinogenesis. Herein, the present inventors found that compounds of the present invention that are scavengers of all electrophiles, reduce the generation of DNA damage and the formation of dysplasia and intramucosal carcinoma in H. pylori-infected INS-GAS mice. Our current data showing that 2-HOBA dampens H. pylori-mediated DNA damage in vitro in gastric epithelial cells and our previous finding that electrophile adducts colocalize with gastric epithelial cells with positive staining for pH2AX support the link between generation of reactive aldehydes and DNA damage, which may ultimately lead to genomic instability and cancer.

The present inventors found that 2-HOBA is detected in the gastric tissues of animals supplemented with this scavenger, supporting the likelihood that it can exert a biological function in the stomach. The present inventors measured less free 2-HOBA in mice infected with H. pylori compared to uninfected animals. Since the LC-MS/MS assay detects only free 2-HOBA, it is speculated that this compound had scavenged high levels of electrophiles in infected mice and was thus less detected.

Our results indicate that H. pylori-infected mice treated with 1 or 3 mg/ml 2-HOBA exhibited a significant reduction of gastritis and dysplasia versus untreated infected mice; we further observed that IMC was not observed in mice receiving 3 mg/ml 2-HOBA. This suggests that scavenging electrophiles reduces not only the formation of precancerous lesions, but also dampens the neoplastic progression. The protective effect on carcinogenesis obtained with 3 mg/ml 2-HOBA, i.e., reduction of total dysplasia by 43% and IMC by 100%, is comparable to the effect seen with 7.5 mg/ml EtHOBA, which reduced dysplasia by 42% with only 1 out 30 mice with IMC. Both EtHOBA and 2-HOBA did not affect H. pylori burden in the stomach, indicating that these electrophile scavengers do not dampen carcinogenesis by an effect on colonization. However, we observed in the present report that both concentrations of 2-HOBA significantly inhibited H. pylori-induced gastritis. The expression of the genes encoding for markers of the innate or T cell specific immune response was also reduced in mice receiving 2-HOBA, thus confirming the histopathology. Similarly, in murine models of hypertension, myocardial ischemic injury, atherosclerosis in hypercholesterolemic Ldlr−/− mice, and systemic lupus erythematosus, the increased expression of pro-inflammatory markers was significantly inhibited by 2-HOBA. In the context of H. pylori infection, chronic inflammation is strongly associated with increased risk for GC; therefore, a compound such as 2-HOBA that reduces both inflammation and neoplastic transformation is of great interest to treat patients.

The present invention shows that compounds of the present invention are useful for prevention of GC in patients infected with H. pylori, by reducing inflammation and oxidative DNA damage that leads to mutagenesis.

### REFERENCES

The following references are incorporated herein by reference in their entirety:
1. Agrawal, A., and Fentiman, I. S. (2008) NSAIDs and breast cancer: a possible prevention and treatment strategy, Int J Clin Pract 62, 444-449.
2. Harris, R. E., Beebe-Donk, J., and Alshafie, G. A. (2007) Reduced risk of human lung cancer by selective cyclooxygenase 2 blockade: results of a case control study, Int J Biol Sci 3, 328-334.
3. Konturek, P. C., Kania, J., Burnat, G., Hahn, E. G., and Konturek, S. J. (2005) Prostaglandins as mediators of COX-2 derived carcinogenesis in gastrointestinal tract, J Physiol Pharmacol 56 Suppl 5, 57-73.
4. Harris, R. E. (2009) Cyclooxygenase-2 (cox-2) blockade in the chemoprevention of cancers of the colon, breast, prostate, and lung, Inflammopharmacology 17, 55-67.
5. Lim, B. J., Jung, S. S., Choi, S. Y., and Lee, C. S. (2010) Expression of metastasis-associated molecules in non-small cell lung cancer and their prognostic significance, Mol Med Report 3, 43-49.
6. Sheehan, K. M., Sheahan, K., O'Donoghue, D. P., MacSweeney, F., Conroy, R. M., Fitzgerald, D. J., and Murray, F. E. (1999) The relationship between cyclooxygenase-2 expression and colorectal cancer, JAMA 282, 1254-1257.
7. Bertagnolli, M. M. (2007) Chemoprevention of colorectal cancer with cyclooxygenase-2 inhibitors: two steps forward, one step back, Lancet Oncol 8, 439-443.
8. Castellone, M. D., Teramoto, H., Williams, B. O., Druey, K. M., and Gutkind, J. S. (2005) Prostaglandin E2 promotes colon cancer cell growth through a Gs-axin-beta-catenin signaling axis, Science 310, 1504-1510.
9. Nakanishi, M., Montrose, D. C., Clark, P., Nambiar, P. R., Belinsky, G. S., Claffey, K. P., Xu, D., and Rosenberg, D. W. (2008) Genetic deletion of mPGES-1 suppresses intestinal tumorigenesis, Cancer research 68, 3251-3259.
10. Elander, N., Ungerbäck, J., Olsson, H., Uematsu, S., Akira, S., and Söderkvist, P. (2008) Genetic deletion of mPGES-1 accelerates intestinal tumorigenesis in APC Min/+ mice, Biochemical and biophysical research communications 372, 249-253.
11. Boutaud, O., Brame, C. J., Salomon, R. G., Roberts, L. J., 2nd, and Oates, J. A. (1999) Characterization of the lysyl adducts formed from prostaglandin H2 via the levuglandin pathway, Biochemistry 38, 9389-9396.
12. Salomon, R. G., Miller, D. B., Zagorski, M. G., and Coughlin, D. J. (1984) Solvent-induced fragmentation of prostaglandin endoperoxides. New aldehyde products from PGH2 and a novel intramolecular 1,2-hydride shift during endoperoxide fragmentation in aqueous solution, J Am Chem Soc 106, 6049-6060.
13. Zagol-Ikapitte, I., Bernoud-Hubac, N., Amarnath, V., Roberts, L. J., 2nd, Boutaud, O., and Oates, J. A. (2004) Characterization of bis(levuglandinyl) urea derivatives as products of the reaction between prostaglandin H2 and arginine, Biochemistry 43, 5503-5510.
14. Boutaud, O., Brame, C. J., Chaurand, P., Li, J., Rowlinson, S. W., Crews, B. C., Ji, C., Marnett, L. J., Caprioli, R. M., Roberts, L. J., 2nd, and Oates, J. A. (2001) Characterization of the lysyl adducts of prostaglandin H-synthases that are derived from oxygenation of arachidonic acid, Biochemistry 40, 6948-6955.
15. Iyer, R. S., Ghosh, S., and Salomon, R. G. (1989) Levuglandin E2 crosslinks proteins, Prostaglandins 37, 471-480.
16. Boutaud, O., Li, J., Zagol, I., Shipp, E. A., Davies, S. S., Roberts, L. J., 2nd, and Oates, J. A. (2003) Levuglandinyl adducts of proteins are formed via a prostaglandin H2 synthase-dependent pathway after platelet activation, The Journal of biological chemistry 278, 16926-16928.
17. Boutaud, O., Andreasson, K. I., Zagol-Ikapitte, I., and Oates, J. A. (2005) Cyclooxygenase-dependent lipid-modification of brain proteins, Brain Pathol 15, 139-142. 18. Jenuwein, T., and Allis, C. D. (2001) Translating the histone code, Science 293, 1074-1080.
19. Esteller, M. (2007) Cancer epigenomics: DNA methylomes and histone-modification maps, Nat Rev Genet 8, 286-298.
20. Fraga, M. F., Ballestar, E., Villar-Garea, A., Boix-Chornet, M., Espada, J., Schotta, G., Bonaldi, T., Haydon, C., Ropero, S., Petrie, K., Iyer, N. G., Perez-Rosado, A., Calvo, E., Lopez, J. A., Cano, A., Calasanz, M. J., Colomer, D., Piris, M. A., Ahn, N., Imhof, A., Caldas, C., Jenuwein, T., and Esteller, M. (2005) Loss of acetylation at Lys16 and trimethylation at Lys20 of histone H4 is a common hallmark of human cancer, Nat Genet 37, 391-400.
21. Zagol-Ikapitte, I., Amarnath, V., Bala, M., Roberts, L. J., 2nd, Oates, J. A., and Boutaud, O. (2010) Characterization of scavengers of gamma-ketoaldehydes that do not inhibit prostaglandin biosynthesis, Chemical research in toxicology 23, 240-250.
22. Zagol-Ikapitte, I., Masterson, T. S., Amarnath, V., Montine, T. J., Andreasson, K. I., Boutaud, O., and Oates, J. A. (2005) Prostaglandin H(2)-derived adducts of proteins correlate with Alzheimer's disease severity, Journal of neurochemistry 94, 1140-1145.
23. Shechter, D., Dormann, H. L., Allis, C. D., and Hake, S. B. (2007) Extraction, purification and analysis of histones, Nat Prot 2, 1445-1457.
24. Bhaskara, S., Knutson, S. K., Jiang, G., Chandrasekharan, M. B., Wilson, A. J., Zheng, S., Yenamandra, A., Locke, K., Yuan, J .-L., Bonine-Summers, A. R., Wells, C. E., Kaiser, J. F., Washington, M. K., Zhao, Z., Wagner, F. F., Sun, Z .-W., Xia, F., Holson, E. B., Khabele, D., and Hiebert, S. W. (2010) Hdac3 is essential for the maintenance of chromatin structure and genome stability, Cancer Cell 18, 436-447.
25. Sanders, M. M. (1978) Fractionation of nucleosomes by salt elution from micrococcal nuclease-digested nuclei, J Cell Biol 79, 97-109.
26. Jiang, T., Zhou, X., Taghizadeh, K., Dong, M., and Dedon, P. C. (2007) N-formylation of lysine in histone proteins as a secondary modification arising from oxidative DNA damage, Proceedings of the National Academy of Sciences of the United States of America 104, 60-65.
27. Fernandez-Alvarez, A., Llorente-Izquierdo, C., Mayoral, R., Agra, N., Bosca, L., Casado, M., and Martin-Sanz, P. (2012) Evaluation of epigenetic modulation of cyclooxygenase-2 as a prognostic marker for hepatocellular carcinoma, Oncogenesis 1, e23.
28. Amarnath, V., Amarnath, K., Davies, S., and Roberts, L. J. n. (2004) Pyridoxamine: an extremely potent scavenger of 1,4-dicarbonyls, Chemical research in toxicology 17, 410-415.
29. Zagol-Ikapitte, I., Amarnath, V., Jadhav, S., Oates, J. A., and Boutaud, O. (2011) Determination of 3-methoxysalicylamine levels in mouse plasma and tissue by liquid chromatography-tandem mass spectrometry: application to in vivo pharmacokinetics studies, Journal of chromatography 879, 1098-1104.
30. Zagol-Ikapitte, I. A., Matafonova, E., Amarnath, V., Bodine, C. L., Boutaud, O., Tirona, R. G., Oates, J. A., Roberts, L. J., 2nd, and Davies, S. S. (2010) Determination of the pharmacokinetics and oral bioavailability of salicylamine, a potent gamma-ketoaldehyde scavenger, by LC/MS/MS, Pharmaceutics 2, 18-29.
31. Murthi, K. K., Friedman, L. R., Oleinick, N. L., and Salomon, R. G. (1993) Formation of DNA-protein cross-links in mammalian cells by levuglandin E2, Biochemistry 32, 4090-4097.
32. Arya, G., and Schlick, T. (2006) Role of histone tails in chromatin folding revealed by a mesoscopic oligonucleosome model, Proceedings of the National Academy of Sciences of the United States of America 103, 16236-16241.
33. Shogren-Knaak, M., Ishii, H., Sun, J .- M., Pazin, M. J., Davie, J. R., and Peterson, C. L. (2006) Histone H4-K16 acetylation controls chromatin structure and protein interactions, Science 311, 844-847.
34. Fuks, F. (2005) DNA methylation and histone modifications: teaming up to silence genes, Curr Opin Genet Dev 15, 490-495.
35. Füllgrabe, J., Kavanagh, E., and Joseph, B. (2011) Histone onco-modifications, Oncogene 30, 3391-3403.
36. Carrier, E .J., et al., Characterization of covalent adducts of nucleosides and DNA formed by reaction with levuglandin. Biochemistry, 2009. 48(45): p. 10775-81.
37. Wang, T. C., et al., Synergistic interaction between hypergastrinemia and Helicobacter infection in a mouse model of gastric cancer. Gastroenterology, 2000. 118(1): p. 36-47.
38. Sierra, J. C., et al., Epidermal growth factor receptor inhibition downregulates Helicobacter pylori-induced epithelial inflammatory responses, DNA damage and gastric carcinogenesis. Gut, 2017.
39. Chaturvedi, R., et al., Activation of EGFR and ERBB2 by Helicobacter pylori results in survival of gastric epithelial cells with DNA damage. Gastroenterology, 2014. 146(7): p. 1739-51 e14.
40. Carrier E J, Zagol-Ikapitte I, Amarnath V, Boutaud O, Oates J A. Levuglandin forms adducts with histone h4 in a cyclooxygenase-2-dependent manner, altering its interaction with DNA. Biochemistry. 2014;53:2436-41
42. Davies S S, Bodine C, Matafonova E, Pantazides B G, Bernoud-Hubac N, Harrison F E, et al. Treatment with a gamma-ketoaldehyde scavenger prevents working memory deficits in hApoE4 mice. J Alzheimers Dis. 2011;27:49-59.
43. Davies S S, May-Zhang L S, Boutaud O, Amarnath V, Kirabo A, Harrison D G. Isolevuglandins as mediators of disease and the development of dicarbonyl scavengers as pharmaceutical interventions. Pharmacol Ther. 2020;205:107418.

It will be understood that various details of the presently disclosed subject matter can be changed without departing from the scope of the subject matter disclosed herein. Furthermore, the foregoing description is for the purpose of illustration only, and not for the purpose of limitation.

We claim:

1. A method of inhibiting the progression of a gastrointestinal cancer in a subject, comprising administering to the subject a levuglandin adduct formation inhibiting amount of a compound of the following formula: 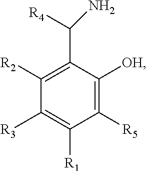
wherein:
R₁ is H, D, D₂, substituted or unsubstituted alkyl, substituted or unsubstituted alkoxy, carboxyl, substituted or unsubstituted aryl, or substituted or unsubstituted cycloalkyl;
R₂ is H, substituted or unsubstituted alkyl, substituted or unsubstituted alkoxy, alkyl-alkoxy-R_{4;}
R₃ is H, halogen, substituted or unsubstituted alkyl, substituted or unsubstituted alkoxy, hydroxyl, nitro;
R₄ H, D, D₂, substituted or unsubstituted alkyl, substituted or unsubstituted alkoxy, carboxyl, substituted or unsubstituted aryl, or substituted or unsubstituted cycloalkyl;
R₅ is H, substituted or unsubstituted alkyl, substituted or unsubstituted alkoxy;
or a pharmaceutical salt thereof.

2. The method of claim 1, wherein the cancer is colorectal cancer.

3. The method of claim 1, wherein R₁ is CH₃ or alkyl.

4. The method of claim 1, wherein one of R₂, R₃, or R₅ is CH₃ or alkyl.

5. The method of claim 1, wherein the compound is of the following formula: 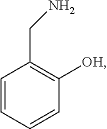
and pharmaceutical salts thereof.

6. The method of claim 1, wherein the compound is 2-hydroxybenzylamine, methyl-2-hydroxybenzylamine, or ethyl-2-hydroxybenzylamine.

7. The method of claim 1, wherein the subject is diagnosed with a H. pylori infection prior to the administration step.

8. A method of mitigating the progression of pre-malignant lesions in a subject in need thereof by administering an effective amount of a compound of the following formula: 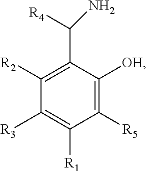
wherein:
R₁ is H, D, D₂, substituted or unsubstituted alkyl, substituted or unsubstituted alkoxy, carboxyl, substituted or unsubstituted aryl, or substituted or unsubstituted cycloalkyl;
R₂ is H, substituted or unsubstituted alkyl, substituted or unsubstituted alkoxy, alkyl-alkoxy-R₄;
R₃ is H, halogen, substituted or unsubstituted alkyl, substituted or unsubstituted alkoxy, hydroxyl, nitro;
R₄ H, D, D₂, substituted or unsubstituted alkyl, substituted or unsubstituted alkoxy, carboxyl, substituted or unsubstituted aryl, or substituted or unsubstituted cycloalkyl;
R₅ is H, substituted or unsubstituted alkyl, substituted or unsubstituted alkoxy;
or a pharmaceutical salt thereof.

9. The method of claim 8, wherein the lesions are in a gastrointestinal tract.

10. The method of claim 8, wherein R₁ is CH₃ or alkyl.

11. The method of claim 8, wherein one of R₂, R₃, or R₅ is CH₃ or alkyl.

12. The method of claim 8, wherein the compound is of the following formula: 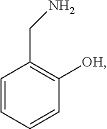
or a pharmaceutical salt thereof.

13. The method of claim 8, wherein the compound is 2-hydroxybenzylamine, methyl-2-hydroxybenzylamine, or ethyl-2-hydroxybenzylamine.

14. A method of treating or inhibiting the progression of gastrointestinal carcinoma in a subject in need thereof, comprising administering an effective H. pylori infection reducing amount of a compound of the following formula: 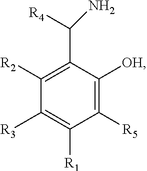
wherein:
R₁ is H, D, D₂, substituted or unsubstituted alkyl, substituted or unsubstituted alkoxy, carboxyl, substituted or unsubstituted aryl, or substituted or unsubstituted cycloalkyl;
R₂ is H, substituted or unsubstituted alkyl, substituted or unsubstituted alkoxy, alkyl-alkoxy-R₄;
R₃ is H, halogen, substituted or unsubstituted alkyl, substituted or unsubstituted alkoxy, hydroxyl, nitro;
R₄ H, D, D₂, substituted or unsubstituted alkyl, substituted or unsubstituted alkoxy, carboxyl, substituted or unsubstituted aryl, or substituted or unsubstituted cycloalkyl;
R₅ is H, substituted or unsubstituted alkyl, substituted or unsubstituted alkoxy;
or a pharmaceutical salt thereof.

15. The method of claim 14, wherein the compound is of the following formula: 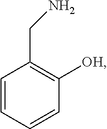
or a pharmaceutical salt thereof.

16. The method of claim 14, wherein the compound is 2-hydroxybenzylamine, methyl-2-hydroxybenzylamine, or ethyl-2-hydroxybenzylamine.

17. The method of claim 14, further including the step of, prior to the administration step, diagnosing the subject as being infected with H. pylori in the gastrointestinal tract.

18. The method of claim 8, wherein the lesion exists in the colon, esophagus, breast, lung, pancreas, gastrointestinal tract, and/or prostate.

19. The method of claim 1, wherein said inhibition lowers levels of LG-histone adduct formation in said subject.

20. A method of preventing gastrointestinal cancer in a subject infected with H. pylori, comprising administering to a subject identified as having a H. pylori infection a compound of the following formula: 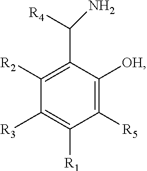
wherein:
R₁ is H, D, D₂, substituted or unsubstituted alkyl;
R₂ is H, substituted or unsubstituted alkyl;
R₃ is H, substituted or unsubstituted alkyl;
R₄ H, D, D₂, substituted or unsubstituted alkyl;
R₅ is H, substituted or unsubstituted alkyl;
or a pharmaceutical salts thereof.
